(19) United States
(12) Patent Application Publication
(10) Pub. No.: US 2014/0094402 A1
(43) Pub. Date: Apr. 3, 2014

(54) Lantibiotic NAI-802, Pharmaceutically Acceptable Salts, Compositions and Uses Thereof

(71) Applicants: Sonia Ilaria Maffioli, Milano (IT); Matteo Simone, Segrate (IT); Paolo Monciardini, Cislago (Varese) (IT); Eleonora Gaspari, Busto Arsizio (Varese) (IT)

(72) Inventors: Sonia Ilaria Maffioli, Milano (IT); Matteo Simone, Segrate (IT); Paolo Monciardini, Cislago (Varese) (IT); Eleonora Gaspari, Busto Arsizio (Varese) (IT)

(73) Assignee: SENTINELLA PHARMACEUTICALS, INC., Princeton, NJ (US)

(21) Appl. No.: 14/007,739

(22) PCT Filed: Mar. 30, 2012
(86) PCT No.: PCT/US2012/031487

§ 371 Date: Dec. 13, 2013

Related U.S. Application Data
(60) Provisional application No. 61/469,158, filed on Mar. 30, 2011.
(60) Provisional application No. 61/533,605, filed on Sep. 12, 2011.
(60) Provisional application No. 61/566,250, filed on Dec. 2, 2011.

(51) Int. Cl. C07K 7/56

(52) U.S. Cl. CPC C07K 7/56
USPC 514/2.9; 530/317; 435/71.3; 435/252.1; 435/252.3; 435/69.1

(57) ABSTRACT

The present invention concerns novel lantibiotic compounds, processes for the isolation and preparation thereof, pharmaceutical compositions containing the same, pharmaceutical acceptable salts thereof, and methods of use of the lantibiotics as antibacterial agents.

### FIELD OF THE INVENTION

The present invention includes novel antibiotic compounds having general formula (I), the processes for their preparation, the key intermediates in said processes, the pharmaceutical acceptable salts and the pharmaceutical compositions containing them as well as their use as therapeutic agents, including antibiotic agents.

### BACKGROUND OF THE INVENTION

The compounds designated as lantibiotics are peptides characterized by the presence of the amino acids lanthionine and/or 3-methyllanthionine. The term lantibiotic thus defines a structural feature of these compounds and not necessarily a common possible use. In fact, some lantibiotics possess antibacterial activity while others are totally devoid of it. Among the lantibiotics possessing antibacterial activity, of particular relevance are those active against methicillin-resistant Staphylococcus aureus (MRSA), which can be of considerable interest in medicine. All the lantibiotics endowed with antibacterial activity described so far, exert their action by interfering with cell wall biosynthesis, through sequestration of a key intermediate in peptidoglycan formation.

The antibacterial lantibiotics can be broadly divided into two groups on the basis of their structures: type-A (also referred to as Class I) lantibiotics are typically elongated, amphiphilic peptides, while type-B (also, referred to as Class II) lantibiotics are compact and globular. Nisin is the typical representative of type A lantibiotic, whereas actagardine and mersacidin belong to the type B lantibiotic subclass. Remarkably, despite differences in shape and primary structure, both nisin-type and mersacidin-type lantibiotics interact with the membrane-bound peptidoglycan precursor lipid II. Furthermore, while the spectrum of antibiotic activity is generally restricted to Gram-positive bacteria, individual members of subclasses A and B greatly vary in their potency. Overall, the structural elements responsible for increased target binding and/or enhanced antibacterial activity in lantibiotics are poorly understood.

Traditionally, lantibiotics have been isolated mostly from the order Firmicutes (low G-C Gram-positive bacteria) and relatively few have been described from the Actinomycetales, the order best known for the ability to produce a large variety of other antibiotics. Actagardine and the recently described 107891 (International Publication Number WO2005/014628) and 97518 (Publication Number EP1481986) are representative lantibiotics produced by the Actinomycetales. These lantibiotics are active in vitro against Methicillin-Resistant Staphylococcus Aureus (MRSA), streptococci and enterococci. S. aureus can cause life-threatening infections and MRSA is of particular clinical significance because it is resistant to all penicillins and cephalosporins and also to multiple other antibiotics; in addition it easily spreads from patient to patient causing outbreaks of infection with important implications for healthcare facilities. Vancomycin resistant enterococci (VRE) are emerging as important hospital-acquired pathogens responsible for severe human infections (such as endocarditis, meningitis and septicemia) posing an increasing therapeutic challenge. Streptococcus pneumoniae and Moraxella catarrhalis are recognized important human pathogens. They are a common cause of respiratory tract infections, particularly otitis media in children and lower respiratory tract infections in the eldery. M. catarrhalis and S. pneumoniae have been recently accepted as the commonest pathogens of the respiratory tract.

Variants and/or derivatives of naturally occurring antibiotics have been long sought after and can be useful in medicine. They can be produced by chemical synthesis or by modification of a natural product, but most structural variations in naturally occurring antibiotics tend to abolish or severely impair their antibacterial activity. This is particularly true in the field of lantibiotics where structure-activity relationships (SAR) are poorly defined, in the absence of molecular details about antibiotic-target interactions. Furthermore, other factors likely to contribute to antibacterial potency are the diffusion rate of the compound to the target, after crossing the thick peptidoglycan layer, and possible interactions with polar, charged and hydrophobic moieties present on the protective external surfaces of the bacterial cell. An additional element rendering unpredictable the outcome of lantibiotic modifications is the existence of unrelated compounds possessing a similar mechanism of action, preventing conclusions drawn from SAR studies on one subtype to be applied to the other.

### DESCRIPTION OF THE INVENTION

The invention encompasses novel antibiotic compounds, methods of making such compounds and their use in the treatment of human or animal subjects, particularly in conditions requiring antibacterial therapy. These and other aspects of the invention are described herein.

The present invention encompasses novel antibiotic compounds, which are lantibiotics, having the general formula (I), the processes for their preparation, the key intermediates in said processes and the pharmaceutical compositions containing them, their pharmaceutically acceptable salts and their use as antibacterial agents.

In an embodiment, the present invention encompasses a lantibiotic substance of microbial origin of general formula (I), pharmaceutical acceptable salts, pharmaceutical compositions and their use as therapeutic agents, for example, as an antibacterial agent.

In an embodiment, the present invention also encompasses a process for preparing lantibiotic derivatives according to formula (I), which comprises culturing Actinoplanes sp. hereinafter identified as Actinoplanes sp. DSM 24057 (deposited on 29 Sep. 2010 with the Deutsche Sammlung von Mikroorganismen und Zellkulturen GmbH (DSMZ) with accession number DSM24057) or a variant or mutant thereof still maintaining the ability to produce said lantibiotic, recovering the lantibiotic according to the present invention from the mycelium and/or from the fermentation broth and isolating the pure substance by chromatographic means.

In an embodiment, the present invention also encompasses a process for preparing lantibiotic derivatives according to formula (I), which comprises culturing Actinoplanes sp. hereinafter identified as Actinoplanes sp. DSM 25201 (deposited on Sep. 22, 2011 with the Deutsche Sammlung von Mikroorganismen und Zellkulturen GmbH (DSMZ) with accession number DSM25201) or a variant or mutant thereof still maintaining the ability to produce said lantibiotic, recovering a lantibiotic according to the present invention from the mycelium and/or from the fermentation broth and isolating the pure substance by chromatographic means.

In an embodiment, the present invention encompasses a process for preparing lantibiotic derivatives according to formula (I), including NAI-802, comprising culturing Actinoplanes sp. 104802, recovering the lantibiotic from the mycelium and/or from the fermentation broth, and isolating the lantibiotic. In an embodiment, the present invention encompasses a process for preparing lantibiotic derivatives according to formula (I), including NAI-802, comprising culturing Actinoplanes sp. 104771, recovering the lantibiotic from the mycelium and/or from the fermentation broth, and isolating the lantibiotic.

In an embodiment, compounds of formula (I) are novel antibacterial agents with a peptide structure containing lanthionine and methyl-lanthionine, having the general formula:

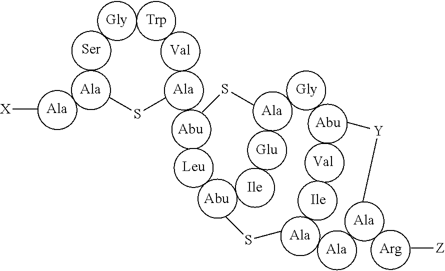

wherein X represents NH₂ or Ala; Y represents —S—, —S(O) (sulfoxide), or —S(O)₂ (sulfone); Z represents OH or NR₁R₂ wherein R₁ and R₂ independently represent:
hydrogen or
an alkyl of 1 to 20 carbon atoms;
an alkenyl of 2 to 20 carbon atoms;
an alkynyl of 2 to 20 carbon atoms;
a cycloalkyl of 3 to 8 carbon atom optionally substituted by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl (C₁-C₄)alkyl, phenoxy, phenoxy portion of the phenoxy-(C₁-C₄)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms;
a phenyl radical optionally substituted by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms
a benzyl radical optionally substituted on the phenyl ring by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms
a naphthyl radical optionally substituted by one or two substituents selected from halo, (C₁-C₄)alkyl optionally substituted by 1 to 3 halogen atoms, and (C1-C4) alkoxy optionally substituted by 1 to 3 halogen atoms
a group of formula

—(CH₂)ₙOR₃
in which n represents an integer from 2 to 8 and R₃ represent
   hydrogen or
   (C₁-C₄) alkyl or
   a cycloalkyl of 3 to 8 carbon atom optionally substituted by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms.
   a phenyl radical optionally substituted by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms
a group of formula

—(CH₂)ₙNR₄R₅
in which n represents an integer from 2 to 8 and R₄ and R₅ independently represent
   hydrogen or
   (C₁-C₄) alkyl or
   a cycloalkyl of 3 to 8 carbon atom optionally substituted by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms.
   phenyl radical optionally substituted by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms
   a benzyl radical optionally substituted on the phenyl ring by one or two substituents independently selected from halo, cyano, (C₁-C₄)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C₁-C₄)alkyl wherein each of phenyl, phenyl portion of the phenyl lower-alkyl, phenoxy, phenoxy portion of the phenoxy-(C1-C4)alkyl group is optionally substituted by one or two substituents selected from halo, cyano, (C₁-C₄)alkyl optionally substituted by 1 to 3 halogen atoms, and (C1-C4) alkoxy optionally substituted by 1 to 3 halogen atoms
   R₄ and R₅ taken together represent a —(CH₂)₃, —(CH₂)₄—, —(CH₂)₂—O—(CH₂)₂, —(CH₂)₂—S—(CH₂)₂ or
   R₄ and R₅ taken together with the adjacent nitrogen atom represent: a piperazine moiety which may be substituted in position 4 with a substituent selected from (C₁-C₄) alkyl, (C₃-C₈) cycloalkyl, pyridyl, benzyl and substituted benzyl wherein the phenyl moiety bears 1 or 2 substituents selected from chloro, bromo, nitro, (C₁-C₄) alkyl and (C₁-C₄) alkoxy.

The term “(C₁-C₄) alkyl” represents straight or branched alkyl chains of from 1 to 4 carbon atoms such as: methyl, ethyl, propyl, 1-methylethyl, butyl, 1-methylpropyl or 1,1-dimethylethyl. The term “(C₃-C₈) cycloalkyl” represents a cycloalkyl group selected from cyclopropyl, cyclobutyl, cyclopentyl, cyclohexyl and cycloheptyl, ciclooctyl. The term “(C₁-C₄) alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

According to one embodiment of the invention, when X is NH₂, Y is —S(O) and Z is OH, the compound of the invention is called NAI-802. In an embodiment, a compound of the invention surprisingly has a net zero charge at physiological pH values, and is still active. In an embodiment, NAI-802 has a net zero charge at physiological pH values.

In an embodiment, a compound is a derivative of NAI-802.

According to another embodiment of the invention, when X is Ala, Y is —S(O) and Z is OH, the compound of the invention is called Ala-NAI-802.

In another embodiment, the present invention describes a compound of formula (I) wherein X is NH₂, Y is —S— and Z is OH, the compound being called deoxy-NAI-802.

In an another embodiment, the present invention describes a compound of formula (I) wherein X is Ala, Y is —S—, and Z is OH, the compound being called deoxy-Ala-NAI-802.

The invention also encompasses novel compounds of general formula formula (I) wherein X represents NH₂ or Ala; Y represents —S—, —S(O)—, —S(O)₂; Z is NR₁R₂ wherein R₁ and R₂ independently are selected as above described. In an embodiment, amidation of a compound is regioselective. By way of a non-limiting example, amidation of NAI-802 is regioselective when only the C-terminal residue reacts while Glu-12 does not react. In an embodiment, a compound made according to this method and specification carries a net charge of +2 at physiological pH, and such a product has unexpectedly improved antibiotic activity as compared to other lantibiotics, such as Actagardine and Michiganin. NAI-802 differs from actagardine in the presence of one alanine residue and one arginine residue at N- and C-terminal positions, respectively. NAI-802 differs from Michiganin in that leucine and isoleucine residues of Michiganin are replaced with valine residues in NAI-802, and the N-terminal serine is replaced by an alanine residue.

In an embodiment, the invention encompasses compounds wherein —NR₁R₂ has the following formula:

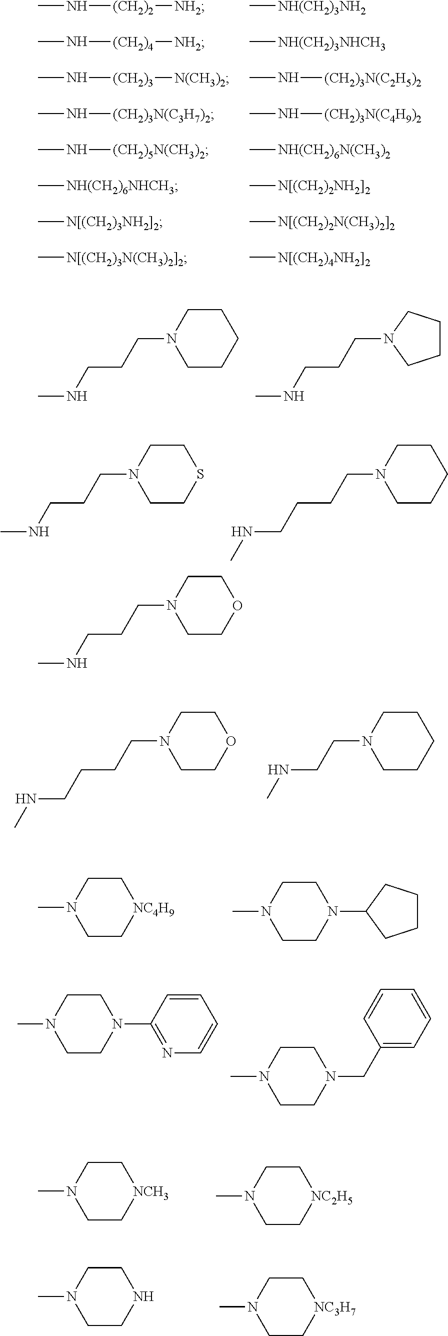

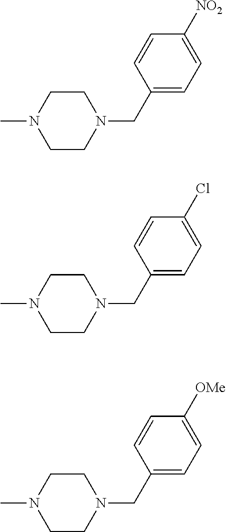 [SMILES: CN1CCN(Cc2ccc([N+](=O)[O-])cc2)CC1 ; CN1CCN(Cc2ccc(Cl)cc2)CC1 ; COc1ccc(CN2CCN(C)CC2)cc1]

In an embodiment, a process is provided for the preparation of the novel compounds having the general formula (I) wherein X is chosen among NH₂ or Ala; Y is chosen among —S—, —S(O)—, —S(O)₂; Z is chosen among OH or NR₁R₂ wherein R₁ and R₂ are defined as above.

In an embodiment, compounds of general formula (I) wherein Z is selected as NR₁R₂ can be obtained and prepared by reacting a compound of formula (I) wherein X is NH₂, Y is —S(O) and Z is OH, with a selected amine of formula HNR₁R₂, wherein R₁ and R₂ are chosen as above.

In an embodiment, a reaction is carried out in the presence of a condensing agent, i.e. in the presence of a solvent. In an embodiment, preferred inert organic aprotic solvents useful for the condensation reaction are those solvents which do not unfavorably interfere with the reaction course and are capable of at least partially solubilizing the starting material, for example compound NAI-802. Solvents optionally can be chosen from among organic amides, ethers of glycols and polyols, phosphoramide derivatives, sulfoxides. Preferably solvents are chosen among: dimethylformamide, dimethoxyethane, hexamethyl phosphoroamide, dimethylsulphoxide, dioxane, N-15 methylpyrrolidone and mixtures thereof. Preferably, dimethylformamide (DMF) is employed. The condensing agent according to the present invention is one suitable for forming amide bonds in organic compounds and, in particular, in peptide synthesis. Representative examples of condensing agents are diisopropylcarbodiimide (DIC), dicyclohexylcarbodiimide (DCC) without or in the presence of hydroxybenzotriazole (HOBT), N,N,N′,N′-tetramethyl-O-(benzotriazol-1-yl)uronium tetrafluoroborate. (TBTU), N,N,N′,N′-tetramethyl-O-(7oxabenzotriazol-1-yl)uranium hexafluorophosphate (HATU), benzotriazolyl-oxy-tris-(dimethylamino)phosphonium hexafluorophosphate (HBTU), benzotriazolyloxy-tris-(pyrrolidino)phosphonium hexafluorophosphate (PyBOP) and (C₁-C₄) alkyl, phenyl or heterocyclic phosphorazidates such as diphenylphosphorazidate, dimorpholyl-phosphorazidate. In an embodiment, a preferred condensing agent is PyBOP. In an embodiment, a condensing agent is generally employed in a slight molar excess, such as from 2.2 to 5; preferably the molar excess of condensing agent is about 2.5 times the molar amount of antibiotic starting compound NAI-802. According to the present method, the amine is normally used in slight molar excess with respect to the compound of formula (I). In an embodiment, a 2- to 10-fold molar excess of the selected amine is used, and in an embodiment, a 4-5 fold molar excess is preferred. When the amine R₁R₂NH is reacted as a corresponding salt, for example the hydrochloride salt, it is necessary to add a suitable base in at least a molar proportion to obtain the free base of the amine R₁R₂NH which reacts with NAI-802. In this case, in an embodiment, an excess of the base is generally preferred. It is convenient to add a salt-forming base to the reaction mixture in an at least equimolecular amount, and preferably in about 1.2 fold molar excess with respect to the amine R₁R₂NH. Examples of such salt-forming bases are tertiary organic aliphatic or alicyclic amines such as trimethylamine, triethylamine (TEA), N-methylpyrrolidine or heterocyclic bases such as picoline and the like, alkali metals (e.g. sodium and potassium) hydrogen carbonates and carbonates. The reaction temperature will vary considerably depending on the specific starting materials and reaction conditions. In general, it is preferred to conduct the amidation reaction at temperature from 0° C. to 50° C. preferably at room temperature. Also, the reaction time varies considerably, depending on the other reaction parameters; in general the condensation is completed in about 2-4-h. However, one of skill in the art, when viewing the disclosure encompassed herein, will understand how to modify these and other reaction parameters in order to obtain a desired product in a desired amount. When the amine R₁R₂NH contains a further primary amino group it might be protected, if necessary, as known in the art, in order to get the desired product. Any typical protecting group of the amino rest, which is resistant to the conditions applied during the process of this invention and may be readily removed under conditions which do not affect the stability of the core portion can be utilized here. Suitable protecting groups of the amino function can be selected, for instance, from the groups described in: T. W. Greene, “Protective Groups in Organic Synthesis”, J. Wiley, N.Y., 1981. In particular, in this case, those protecting groups, which are formed by acylating the amino moiety, are preferred. The protecting groups employed in the process herein described are those generally employed in peptides synthesis. A deprotection step is then necessary to obtain the desired final product. Generally, the reaction course is monitored by HPLC according to methods known in the art. On the basis of the results of this assays it will be possible to evaluate the reaction course and decide when to stop the reaction and start working up the reaction mass according to per se known techniques which include, for instance, precipitation by addition of non-solvents, extraction with solvents, in conjunction with further common separation operations and purification, e.g. by column chromatography.

In an embodiment, according to the full scope of the compositions and methods of the invention as encompassed herein, a series of compounds can be prepared, as summarized in Table 1.

TABLE 1

| | —NR₁R₂ |
|---|---|
| 1 | 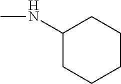 |
| 2 | 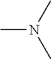 |
| 3 | 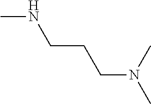 |
| 4 | 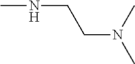 |
| 5 | 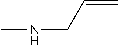 |
| 6 | 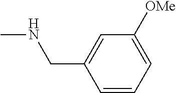 |
| 7 | 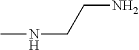 |
| 8 | 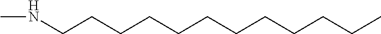 |
| 9 | 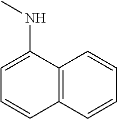 |
| 10 | 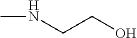 |
| 11 | 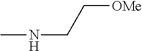 |
| 12 | 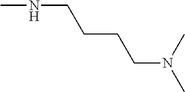 |
| 13 | 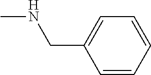 |
| 14 | 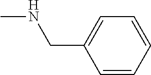 |
| 20 | 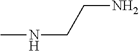 |
| 22 | 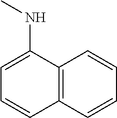 |
| 24 | 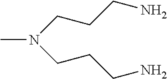 |
| 25 | 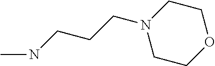 |
| 26 | 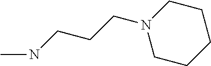 |
| 27 | 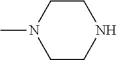 |
| 28 | 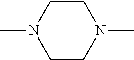 |
| 29 | 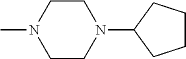 |
| 30 | 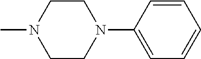 |
| 31 | 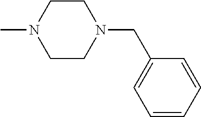 |

In an embodiment, particularly preferred is the compound according to the present invention where X is NH₂, Y is S(O), Z is —NHCH₂CH₂NH₂.

Compounds of general formula (I) possess acid and/or basic functions, they are capable of forming salts with suitable bases or acids according to known procedures and it may exist also in the form of inner salt. The antibiotics, when obtained in the acid form or in the form of inner salt, may be converted into a corresponding non-toxic pharmaceutically acceptable salt with bases. Suitable salts include the alkali and alkaline earth metal salts, typically the sodium, potassium, calcium and magnesium salts, and the ammonium and substituted ammonium salts. Representative substituted ammonium salts include primary, secondary or tertiary (C₁-C₄) alkylammonium and hydroxy (C₁-C₄) alkylammonium salts and, according to an embodiment of the present invention, the benzathine, procaine, hydrabamine and similar water insoluble, non-toxic, pharmaceutically acceptable salts. Another preferred class of salts of the compound of the present invention is represented by the basic addition salts with basic amino acids such as arginine or lysine, or aminosugars such as glucosamine and the like.

The alkali and alkaline earth metal salts are prepared according to the usual procedures commonly employed for preparing metal salts. As an example, antibiotic NAI-802 in the acid form or in the inner salt form, is dissolved into the minimum amount of a suitable solvent, typically a lower alkanol, or a lower alkanol water mixture, the stoichiometric amount of a suitable selected base is gradually added to the obtained solution and the obtained salt is precipitated by the addition of a non-solvent. The alkali or alkaline earth metal salt, which forms are then recovered by filtration or evaporation of the solvents.

Alternatively, these salts can be prepared in a substantially anhydrous form through lyophilization; in this case aqueous solutions containing the desired salts, resulting from the salification of compound NAI-802 with a suitably selected alkali or alkaline earth metal carbonate or hydroxide in such a quantity as to obtain a pH comprised between and are filtered from any non soluble and lyophilized.

The organic ammonium salts can be prepared according to the above procedure by adding the properly selected amine to a solution of NAI-802 compound in a suitable solvent and then evaporating off the solvent and the excess of the amine reagent or by lyophilizing the concentrate solution.

The addition salts of NAI-802 compound with acids can be also prepared. Representative and suitable acid addition salts of the compounds of the invention include those salts formed by standard reaction with both organic and inorganic acids such as, for example, hydrochloric, hydrobromic, sulfuric, phosphoric, acetic, trifluoroacetic, trichloroacetic, succinic, citric, ascorbic, lactic, maleic, fumaric, palmitic, cholic, pamoic, mucic, glutamic, camphoric, glutaric, glycolic, phthalic, tartaric, lauric, stearic, salicylic, methanesulfonic, benzenesulfonic, sorbic, picric, benzoic, cinnamic and the like acids. The addition salts of NAI-802 compound with acids can be prepared in a substantially analogues manner as that employed for the preparation of the salts with bases but using the appropriately selected acid as reagent in the place of the base.

As known in the art, the salt formation with either pharmaceutically or non-pharmaceutically acceptable acids may be used as a convenient purification technique. After formation and isolation, the salt form of a compound of formula (I) can be transformed into the corresponding non-salt or into a pharmaceutically acceptable salt. In some instances the acid addition salt of a compound of formula (I) is more soluble in water and hydrophilic solvents and has an increased chemical stability. Good solubility and stability in water or hydrophilic solvents of an active compound are in general appreciated in the art, for the preparation of suitable pharmaceutical compositions for the administration of the medicament. However, in view of the similarity of the properties of the compounds of formula (I) with their salts, what is said in the present application when dealing with the biological activities of the non-salt compounds of formula (I) applies also to their pharmaceutically acceptable salts, and vice versa.

In an embodiment, the compounds of the present invention can be administered orally, topically or parenterally, the preferred route of administration depending on the treatment to be carried out. Depending on the route of administration, these compounds can be formulated into various dosage forms. Preparations for oral administration may be in the form of capsules, tablets, liquid solutions or suspensions. As known in the art, the capsules and tablets may contain in addition to the active ingredient conventional excipients such as diluents e.g. lactose, calcium phosphate, sorbitol and the like lubricants e.g. magnesium stearate, talc, polyethylene glycol, binding agents, e.g. polyvinylpyrrolidone, gelatin, sorbitol, tragacanth, acacia, flavoring agents, and acceptable disintegrating and wetting agents. The liquid preparations generally in the form of aqueous or oily solutions or suspensions may contain conventional additives such as suspending agents. For topical use, the compounds of formula (I) of the present invention may also be prepared in suitable forms for absorption through the mucous membranes of the nose and throat or bronchial tissues and may conveniently take the form of liquid sprays or inhalants lozenges or throat paints. For medication of the eyes, the preparation may be presented in liquid or semi-liquid form. Topical applications may be formulated in hydrophobic or hydrophilic bases as ointments, creams, lotions, paints, or powders. For rectal administration the compounds of formula (I) of the invention are administered in the form of suppositories admixed with conventional vehicles, such as, for example, cocoa butter, wax, spermaceti or polyethyleneglycols and their derivatives. Compositions for injection may take such forms as suspensions, solutions, or emulsions in oily or aqueous vehicles, and may contain formulation agents such as suspending, stabilizing and/or dispersing agents. Alternatively, the active ingredient may be in powder form for reconstitution at the time of delivery with a suitable vehicle, such as sterile water. The amount of active principle to be administered depends on various factors such as the size and conditions of the subject to be treated, the route and frequency of administration, and the causative agent involved.

In an embodiment, the compounds of the invention are generally effective at a dosage comprised between about 1 and 30 about 40 mg of active ingredient per Kg of body weight. Depending on the characteristics of the specific compound, the infection and the patients, the effective dose can be administered in a single administration per day or divided in 2 to 4 administrations per day. In an embodiment, particularly desirable compositions are those prepared in the form of dosage units containing from about 30 to about 500 mg per unit.

In an embodiment, compounds of the present invention can also be employed in combination with other drugs, being that another antibacterial agent or an agent intended to treat a second symptom or the cause of a different condition. For example, the antibacterial agents that can be used in conjunction with the compounds of the present invention include but are not limited to penicillins, cephalosporins, aminoglycosides, glycopeptides, rifamycins, lipopeptides, aminoglycosides. Therefore, compositions of the compounds of the present invention with other approved drugs fall also within the scope of the present invention.

In an embodiment, novel compounds of formula (I) according with the present invention, including salts, formulation and compositions thereof, can be effectively employed as the active ingredients of the antimicrobial preparations used in human or animal medicine for the prevention and treatment of infectious diseases caused by gram positive aerobic and anaerobic bacteria, such as Enterococcus sp., Streptococcus sp., Staphylococcus sp, Clostridium sp., including strains resistant to commonly used antibiotics.

In an embodiment, also encompassed herein is the use of a compound or composition thereof disclosed herein for the manufacture of a medicament for use in a specific method of treatment or prophylaxis of the human or animal body.

Thus, in an embodiment, compounds of the invention are used for the prevention and treatment of infectious diseases caused by gram positive aerobic and anaerobic bacteria, such as Enterococcus sp., Streptococcus sp., Staphylococcus sp., Clostridium sp., including strains resistant to commonly used antibiotics.

According to one aspect of the invention, compounds of formula (I) are added to animal feed. In an embodiment, such as aspect is preferably accomplished by preparing an appropriate feed premix containing the active compound in an effective amount and incorporating the premix into the complete ration. Alternatively, an intermediate concentrate or feed supplement containing the active ingredient can be blended into the feed.

The way in which such feed premixes and complete rations can be prepared and administered are described in reference books such as “Applied Animal Nutrition”, W.H. Freedman and CO., S. Francisco, U.S.A., 1969 or “Livestock Feeds and Feeding” 0 and B books, Corvallis, Ore., U.S.A., 1977.

### BRIEF DESCRIPTION OF THE DRAWINGS

FIG. 1 and represents mass spectrum of antibiotic NAI-802 showing a doubly protonated ion at m/z 1059 and a triple protonated ion at m/z 707.

FIG. 2 (full-scan low resolution spectrum) represents mass spectrum of antibiotic NAI-802 showing a doubly protonated ion at m/z 1059 and a triple protonated ion at m/z 707.

FIG. 3 represents the UV spectrum of antibiotic NAI-802 dissolved in Acetonitrile:water 1:1.

FIG. 4 represents the ¹H-NMR spectrum recorded in the mixture Acetonitrile-d₃:D₂0-H₂O at 25° C. on a Bruker AMX 600 spectrometer.

FIG. 5 represents the HSQC NMR spectrum recorded in the mixture Acetonitrile-d₃:D₂0 at 25° C. on a Bruker AMX 600 spectrometer.

FIG. 6 represents the HMBC NMR spectrum recorded in the mixture Acetonitrile-d₃:D₂0 at 25° C. on a Bruker AMX 600 spectrometer.

FIG. 7 (full-scan low resolution spectrum) represents mass spectrum of antibiotic Ala-NAI-802 showing a doubly protonated ion at m/z 1095.

FIG. 8 (full-scan low resolution spectrum) represents mass spectrum of antibiotic deoxy-NAI-802 showing a doubly protonated ion at m/z 1050.

FIG. 9 (full-scan low resolution spectrum) represents mass spectrum of NAI-802 monoamide with ethylendiamine (compound 7 of table 1) showing a doubly protonated ion at m/z 1080 and a triple protonated ion at m/z 720.

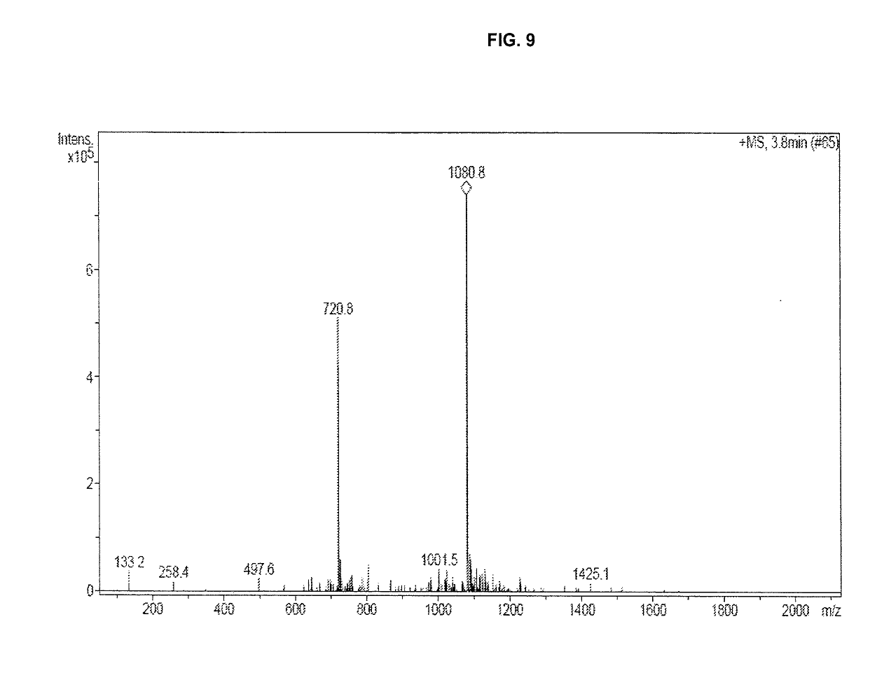

FIG. 10 represents a chromatogram for the first step of purification of NAI-802.

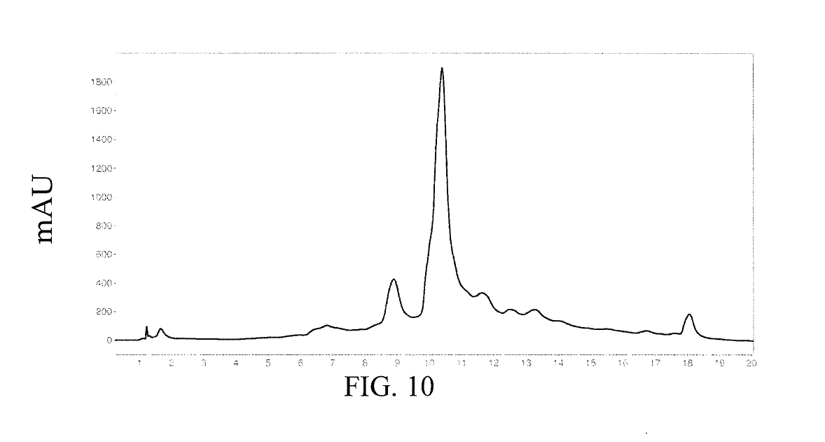

FIG. 11 represents a chromatogram for the second step of purification of NAI-802.

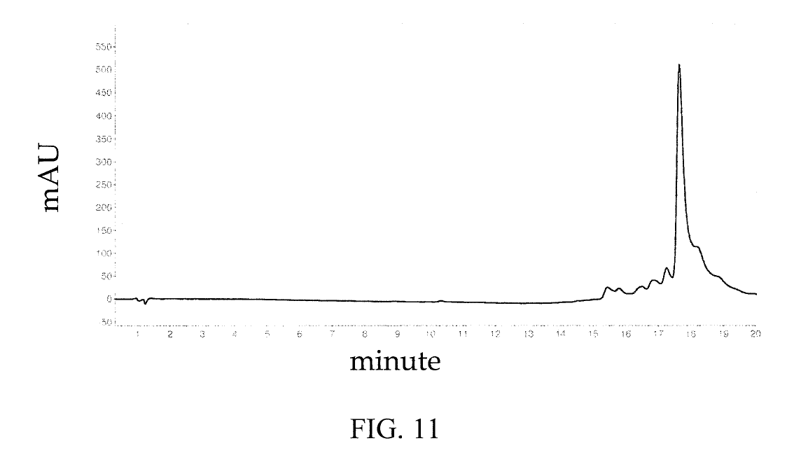

FIG. 12 represents the structure of NAI-802.

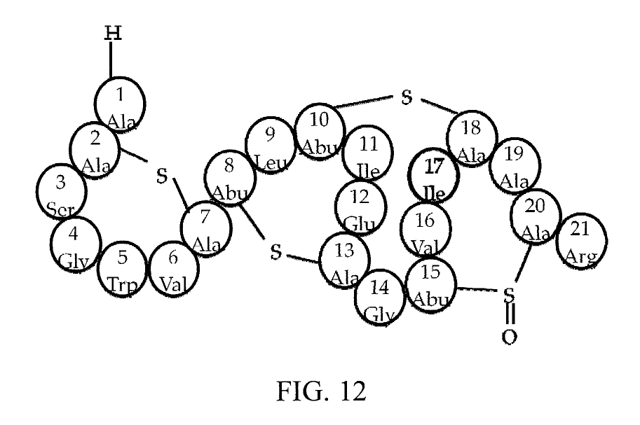

### Strains and Fermentation

The production of lantibiotic NAI-802 is achieved by cultivating an Actinoplanes sp. strain capable of producing it, i.e. Actinoplanes sp. DSM24057, DSM25201, or a variant or mutant of either maintaining the ability to produce lantibiotic NAI-802, isolating the resulting lantibiotic from the whole culture broth and/or from the separated mycelium and/or from the filtered fermentation broth, and purifying the isolated lantibiotic by chromatographic means. In an embodiment, NAI-802, or a derivative thereof, is isolated and purified from Actinoplanes sp. 104802. In an embodiment, NAI-802, or a derivative thereof, is isolated and purified from Actinoplanes sp. 104771.

According to one preferred embodiment the production of lantibiotic NAI-802 is carried out under aerobic conditions in an aqueous nutrient medium containing easy digestible or usable sources of carbon, nitrogen, and inorganic salts. Many of the nutrient media usually employed in fermentation field can be used, however preferred carbon sources are starch, dextrin, glucose, maltose, glycerol, and the like. Preferred nitrogen sources are soybean meal, peptone, meat extract, hydrolyzed casein, tryptone, corn steep liquor, cottonseed meal, yeast extract, and the like.

Soluble salts capable of yielding sodium, potassium, iron, zinc, cobalt, magnesium, calcium, ammonium, chloride, carbonate, sulphate, phosphate, nitrate, and the like ions can be incorporated in certain media.

Preferably, the strain producing antibiotic 802 is pre-cultured in a fermentation tube or in a shake flask, then the culture is used to inoculate jar reactors for fermentation for the production of substantial quantities of substances. The medium used for the pre-culture can be the same as that employed for larger fermentations, but other media can also be employed.

According to one preferred aspect, Actinoplanes sp. DSM24057 strain is grown on S1 plates (detailed information are described in Experimental part) where the strain forms dark orange colonies with whitish aerial mycelium. A brown-green pigment is released in the medium with ageing of the cultures. In another aspect, Actinoplanes sp. DSM25201 strain is grown on Si plates and cultured as above for DSM24057.

The temperature for growing strain Actinoplanes sp. DSM24057 or Actinoplanes sp. DSM25201 producing antibiotic NAI-802 is 26-35° C., preferably 28-32° C. During the fermentation, antibiotic NAI-802 production can be monitored by bioassay on susceptible microorganisms and/or by HPLC analyses. Maximum production of antibiotic NAI-802 generally occurs after 72 hours and before 192 hours of fermentation.

In an embodiment, antibiotic NAI-802 is thus produced by cultivating Actinoplanes sp. DSM24057, Actinoplanes sp. DSM25201 or a variant or mutant of either capable of producing antibiotic 802, and it is found in the culture broths and/or in the mycelium. In an embodiment, lantibiotic is about equally distributed between the culture broth and the mycelium.

Actinoplanes sp. DSM24057 16S rRNA Gene Sequence

The partial sequence of the 16 rRNA gene (16S rDNA), i.e 920 nucleotides, of strain Actinoplanes sp. DSM24057 is reported in SEQ ID NO 1. This sequence is compared with those deposited in public databases, and is found to be related to the 16S rRNA gene sequences of various Actinoplanes strains.

As with other microorganisms, the characteristics of strain producing antibiotic 104802 are subject to variation. For example, artificial variants and mutants of the strain can be obtained by treatment with various known mutagens, such as U.V. rays, and chemicals such as nitrous acid, N-methyl-N′-nitro-N-nitrosoguanidine, and many others. All natural and artificial variants and mutants of strain Actinoplanes sp. DSM24057 are capable of producing antibiotic NAI-802.

| SEQ ID NO: 1 | |
|---|---|
| (16S rRNA gene of strain Actinoplanes sp. DSM24057): | |
| 1 | ATGGCTCAGG ACGAACGCTG GCGGCGTGCT TAACACATGC AAGTCGAGCG |
| 51 | GAAAGGCCCT TCGGGGTACT CGAGCGGCGA ACGGGTGAGT AACACGTGAG |
| 101 | TAACCTGCCC CAGACTTTGG GATAACCCTC GGAAACGGGG GCTAATACCG |
| 151 | GATATGACCT TCGGCCGCAT GGTTGTTGGT GGAAAGTTTT TCGGTTTGGG |
| 201 | ATGGACTCGC GGCCTATCAG CTTGTTGGTG GGGTAATGGC CTACCAAGGC |
| 251 | GACGACGGGT AGCCGGCCTG AGAGGGCGAC CGGCCACACT GGGACTGAGA |
| 301 | CACGGCCCAG ACTCCTACGG GAGGCAGCAG TGGGGAATAT TGCACAATGG |
| 351 | GCGGAAGCCT GATGCAGCGA CGCCGCGTGA GGGATGACGG CCTTCGGGTT |
| 401 | GTAAACCTCT TTCAGCAGGG ACGAAGCGTA AGTGACGGTA CCTGCAGAAG |
| 451 | AAGCGCCGGC CAACTACGTG CCAGCAGCCG CGGTAAGACG TAGGGCGCGA |
| 501 | GCGTTGTCCG GATTTATTGG GCGTAAAGAG CTCGTAGGCG GCTTGTCGCG |
| 551 | TCGTCTGTGA AAACTTGGGG CTCAACCCCA AGCTTGCAGT CGATACGGGC |
| 601 | AGGCTAGAGT TCGGTAGGGG AGACTGGAAT TCCTGGTGTA GCGGTGAAAT |
| 651 | GCGCAGATAT CAGGAGGAAC ACCGGTGGCG AAGGCGGGTC TCTGGGCCGA |
| 701 | TACTGACGCT GAGGAGCGAA AGCGTGGGGA GCGAACAGGA TTAGATACCC |
| 751 | TGGTAGTCCA CGCTGTAAAC GTTGGGCGCT AGGTGTGGGG GACCTCTCCG |
| 801 | GTCTTCTGCG CCGCAGCTAA CGCATTAAGC GCCCCGCCTG GGGAGTACGG |
| 851 | CCGCAAGGCT AAAACTCAAA GGAATTGACG GGGGCCCGCA CAAGCGGCGG |
| 901 | AGCATGCGGA TTAATTCGAT |

### Extraction and Purification of Antibiotic NAI-802

Compound NAI-802, i.e. compound of formula I wherein X is NH₂, Y is —S(O) and Z is OH, is distributed both in the mycelium and in the filtered fraction of the fermentation broth. The harvested broth is processed to separate the mycelium from the supernatant of the fermentation broth and the mycelium is extracted with a water-miscible solvent to obtain a solution containing the antibiotic, after removal of the spent mycelium. This mycelium extract is then processed separately or in pool with the supernatant according to the procedures reported hereafter for the supernatant fraction. When the water-miscible solvent would cause interferences with the operations for recovering the antibiotic from the mycelium extract, the water-miscible solvent is removed by distillation or is diluted with water to a non-interfering concentration.

As used herein, the term “water-miscible solvent” refers to solvents that, at the conditions of use, are miscible with water in a reasonably wide concentration range. Examples of water-miscible organic solvents that can be used in the extraction of the compounds of the invention are: lower alkanols, e.g. (C1-C3) alkanols such as methanol, ethanol, and propanol, phenyl (C1-C3) alkanols such as benzyl alcohol; lower ketones, e.g. (C1-C4) ketones such as acetone and ethyl methyl ketone; cyclic ethers such as dioxane and tetrahydrofuran; glycols and their products of partial etherification such as ethylene glycol, propylene glycol, and ethylene glycol monomethyl ether, lower amides such as dimethylformamide and diethylformamide; acetic acid dimethylsulfoxide and acetonitrile.

The recovery of the compound from the supernatant of the fermentation broth of the producing microorganism is conducted according to known per se techniques which include extraction with solvents, precipitation by adding non-solvents or by changing the pH of the solution, by partition chromatography, reverse phase partition chromatography, ion exchange chromatography, molecular exclusion chromatography and the like or a combination of two or more of said techniques. A procedure for recovering the compounds of the invention from the filtered fermentation broth includes extraction of antibiotic NAI-802 with water-immiscible organic solvents, followed by precipitation from the concentrated extracts, possibly by adding a precipitating agent.

As used herein, the term “water-immiscible solvent” refers to solvents that, at the conditions of use, are slightly miscible or practically immiscible with water in a reasonably wide concentration range, suitable for the intended use. Examples of water-immiscible organic solvents that can be used in the extraction of the compounds of the invention from the fermentation broth are: alkanols of at least four carbon atoms which may be linear, branched or cyclic such as n-butanol, 1-pentanol, 2-pentanol, 3-pentanol, I-hexanol, 2-hexanol, 3-hexanol, 3,3-dimethyl-1-butanol, 4-methyl-1-pentanol, 3-methyl-1-pentanol, 2,2-dimethyl-3-pentanol, 2,4-dimethyl-3-pentanol, 4,4-dimethyl2-pentanol, 5-methyl-2-hexanol, 1-heptanol, 2-heptanol, 5-methyl-1-hexanol, 2-ethyl-1-hexanol, 2-methyl-3-hexanol, 1 octanol, 2-octanol, cyclopentanol, 2-cyclopentylethanol, 3-cyclopenthyl-1-propanol, cyclohexanol, cycloheptanol, cyclooctanol, 2,3-dimethyl-cyclohexanol, 4-ethylcyclohexanol, cyclooctylmethanol, 6-methyl-5-hepten-2-01, 1-nonanol, 2nonanol, 1-decanol, 2-decanol, and 3-decanol; ketones of at least five carbon atoms such as methylisopropylketone, methylisobutylketone, methyl-n-amylketone, methylisoamylketone and mixtures thereof.

As known in the art, product extraction from the filtered fermentation broth may be improved by adjusting the pH at an appropriate value, and/or by adding a proper organic salt forming an ion pair with the antibiotic, which is soluble in the extraction solvent. As known in the art, phase separation may be improved by salting the aqueous phase.

When, following an extraction, an organic phase is recovered containing a substantial amount of water, it may be convenient to azeotropically distill water from it. Generally, this requires adding a solvent capable of forming minimum azeotropic mixtures with water, followed by the addition of a precipitating agent to precipitate the desired product, if necessary. Representative examples of organic solvents capable of forming minimum azeotropic mixtures with water are: n-butanol, benzene, toluene, butyl ether, carbon tetrachloride, chloroform, cyclohexane, 2,5-dimethylfuran, hexane, and mxylenei the preferred solvent being n-butanol. Examples of precipitating agents are petroleum ether, lower alkyl ethers, such as ethyl ether, propyl ether, and butyl ether, and lower alkyl ketones such as acetone.

According to a preferred procedure for recovering antibiotic NAI-802, the filtered fermentation broth can be contacted with an adsorption matrix followed by elution with a polar, water miscible solvent or a mixture thereof, concentration to an oily residue under reduced pressure, and precipitation with a precipitating agent of the type already mentioned above.

Examples of adsorption matrixes that can be conveniently used in the recovery of the compounds of the invention, are polystyrene or mixed polystyrene-divinylbenzene resins (e.g. M112 or 8112, Dow Chemical Co.; Amberlite® XAD2 or XAD4, Rohm & Haasi Diaion HP 20, Mitsubishi), acrylic resins (e.g. XAD7 or XAD8, Rohm & Haas), polyamides such as polycaprolactames, nylons and cross-linked polyvinylpyrrolidones (e.g. Polyamide-CC 6, Polyamide-8C 6, Polyamide-CC 6.6, Polyamide-CC 6AC and Polyamide-SC 6AC, Macherey-Nagel & Co., Germany; PA 400, 'M. Woelm AG, Germany); and the polyvinylpirrolidone resin PVPCL, (Aldrich Chemie GmbH & Co., KG, Germany) and controlled pore cross-linked dextrans (e.g. Sephadex® LH-20, Pharmacia Fine Chemicals, AB). Preferably polystyrene resins are employed, particularly preferred being the Diaion HP 20 resin. In the case of polystyrene resins, polystyrenedivinylbenzene resins, polyamide resins or acrylic resins a preferred eluent is a water-miscible solvent or its aqueous mixtures. The aqueous mixtures can contain buffers at appropriate pH value.

The successive procedures for the isolation and purification of the antibiotic may be carried out on the pooled extracts from the broth supernatant and from the mycelium. For example, when the portion of the antibiotic product contained in the filtered fermentation broth or supernatant is recovered by absorption on an absorption resin and the portion of the antibiotic product contained in the mycelium is extracted therefrom with a water-miscible solvent, followed by adsorption onto an absorption resin, the eluted fractions from each of the two sets of absorption resins are combined, optionally after concentration, and then further processed as a unitary crop. Alternatively, when the two sets of absorption resins utilized for the separate extraction stages are of the same type and have the same functional characteristics, they are pooled together and the mixture may be submitted to a unitary elution step, for instance, with a water-miscible solvent or a mixture thereof with water. In any case, whatever the procedure adopted for recovering the antibiotic NAI-802, the successive purification step is usually carried out on the mixture of the crude materials resulting from the combination of the separate extraction stages.

Purification of the crude antibiotic NAI-802 can be accomplished by any of the known per se techniques but is preferably conducted by means of chromatographic procedures.

Examples of these chromatographic procedures are those reported in relation to the recovery step and include also chromatography on stationary phases such as silica gel, alumina, activated magnesium silicate and the like or reverse phase chromatography on silanized silica gel having various functional derivatizations, and eluting with water miscible solvents or aqueous mixture of water-miscible solvents of the kind mentioned above.

For instance, preparative HPLC or medium or low pressure liquid chromatography may be employed, using RP-8 or RP-18 as stationary phase and a mixture of HCOONH₄ buffer (or TFA 0.1%): CH₃CN as eluting system. The active fractions recovered from the purification step are pooled together, concentrated under vacuum, precipitated by addition of a precipitating agent of the kind mentioned above and dried or lyophilized in single or iterative rounds. In the case the product contains residual amounts of ammonium formate or other buffering salts, these may be removed by absorption of the antibiotic NAI-802 on solid phase extraction column, for instance a reverse phase resin column such as SPE Superclean LCP 18 Supelco (Belle fonte PA, USA) followed by washing with distilled water and elution with an appropriate aqueous solvent mixture, e.g. methanol:water. The antibiotic is then recovered by removing the elution solvents.

In an embodiment, purification and isolation of lantibiotic using two purification steps results in a recovery of total lantibiotic of about 50%.

Accordingly, purified antibiotic NAI-802 dried preparations are obtained as a white powder. As usual in this art, the production as well as the recovery and purification steps may be monitored by a variety of procedures including inhibitory assay against susceptible microorganisms, HPLC or HPLC coupled with mass spectrometry.

HPLC method 1: A preferred analytical HPLC technique is performed on a Shimadzu instrument (LC 2010A-HT liquid chromatograph, Shimadzu Corporation, Japan) equipped with a column LiChrosphere RP18, 5μ (125×4.6 mm) eluted at 1 ml/min flow rate and at 50° C. temperature.

Elution is with a multistep program: Time=0 (10% phase B); Time=20 min (50% Phase B); Time=21 min (80% of phase B); Time=25 min (80% of phase B); Time=26 min (10% of phase B); Time=35 min (10% of phase B). Phase A is trifluoroacetic acid 0.1% in water (v/v) and Phase B is acetonitrile. UV detector is at 230 nm and 270 nm. In these analytical HPLC conditions the antibiotic NAI-802 shows retention times of 18.6 min.

HPLC method 2: A preferred analytical HPLC-MS technique is performed on a Agilent 1100 series liquid chromatograph equipped with a column Ascentis express Supelco RP18, 2.7μ (50×4 6 mm) eluted at 1 ml/min flow rate and at 40° C. temperature. Elution is with a multistep program: Time=0 (5% phase B); Time=6 min (95% Phase B); Time=7 min (100% phase B); Time=7.2 min (5% phase B); Time=10 min (5% phase B). Phase A is trifluoroacetic acid 0.05% in water (v/v) and phase B is trifluoroacetic acid 0.05% in acetonitrile (v/v). UV detector is at 220 nm.

The effluent from the column is split in a 50:50 ratio and one part (500 μL/min) is diverted to photodiode array detector. The remaining 500 μL/min are diverted to the ESI interface of a Bruker Esquire3000 plus ion trap mass spectrometer.

The mass spectrometric analysis is performed under the following conditions: sample inlet conditions: sheat gas (N₂) 50 psi; dry gas 10 L/min; capillary heater 365° C.; sample inlet voltage settings: polarity: positive; capillary voltage −4000V; end plate offset −500V; Scan conditions: maximum ion time 200 ms; ion time 5 ms; full micro scan 3; segment: duration 10 min, scan events positive (100-2400 m/z). In these analytical HPLC-MS conditions the antibiotic NAI-802 shows retention times of 4.1 min.

### Physico-Chemical Characteristics of Antibiotic NAI-802

NAI-802 has a molecular weight of 2118. An amidation reaction (PhCH₂NH₂) demonstrated two —COOH reactive moieties on NAI-802. Edman degratdation (Ph-NCS) demonstrated the presence of an alanine at the N-terminal position of NAI-802. Ethanethiol (EtSH) analysis at pH 7.0 demonstrated the absence of both dehydroalanine (DHA) and dehydrobutyrine (DHB) from NAI-802. EtSH analysis at pH 10.0 revealed that NAI-802 tested positive for 3-4-S—, —S—S—, moieties. Hydrolysis in 6N HCl was used to elucidate the amino acid composition, as described in detail elsewhere herein.

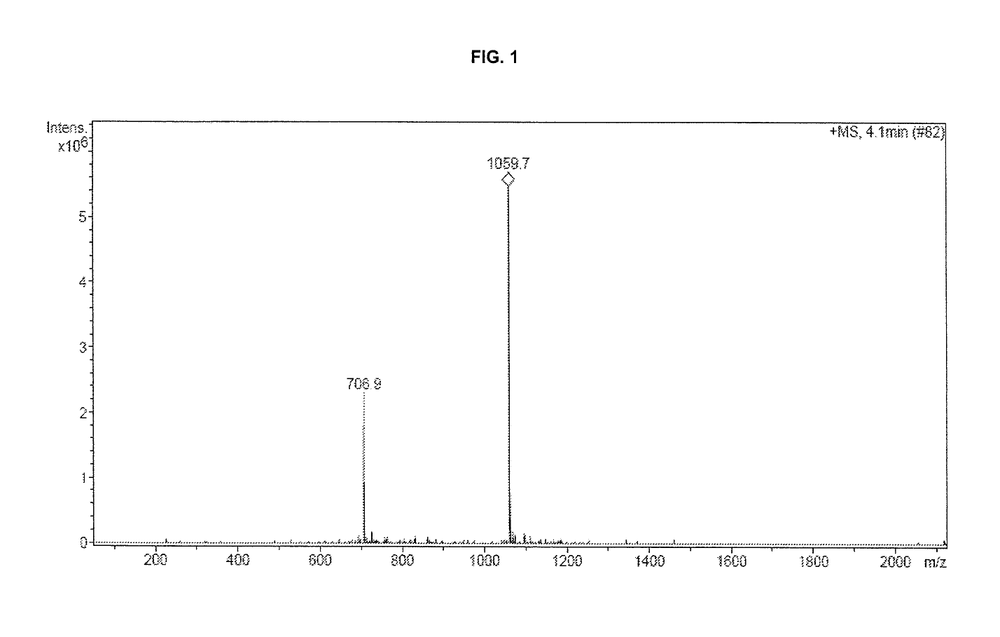

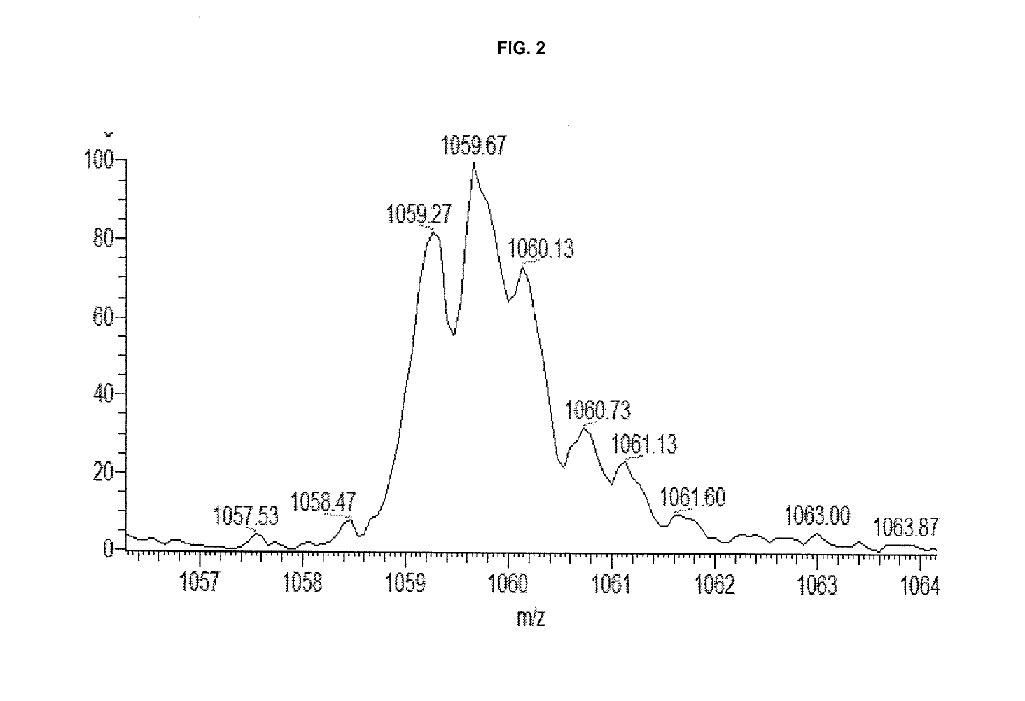

A) Mass spectrometry: in MS experiments on a Thermofinnigan LCQ deca instrument fitted with an electrospray source, using Thermofinnigan calibration mix, antibiotic NAI-802 gives a doubly protonated ion at 1059 m/z. MS/MS analysis of the double charged ion is performed with the observed main fragmentations: monocharged 673, 1445, 1856 and double charged 1050 m/z. The electrospray conditions are: Spray Voltage: 4.7 kV; Capillary temperature: 220° C.; Capillary Voltage: 3 V; Infusion mode 10 μL/min. Spectra are recorded from a 0.2 mg/ml solution in methanol/water 80/20 (v/v) with trifluoroacetic acid 0.1% and are reported in FIG. 1 and FIG. 2 (full-scan low resolution spectrum).

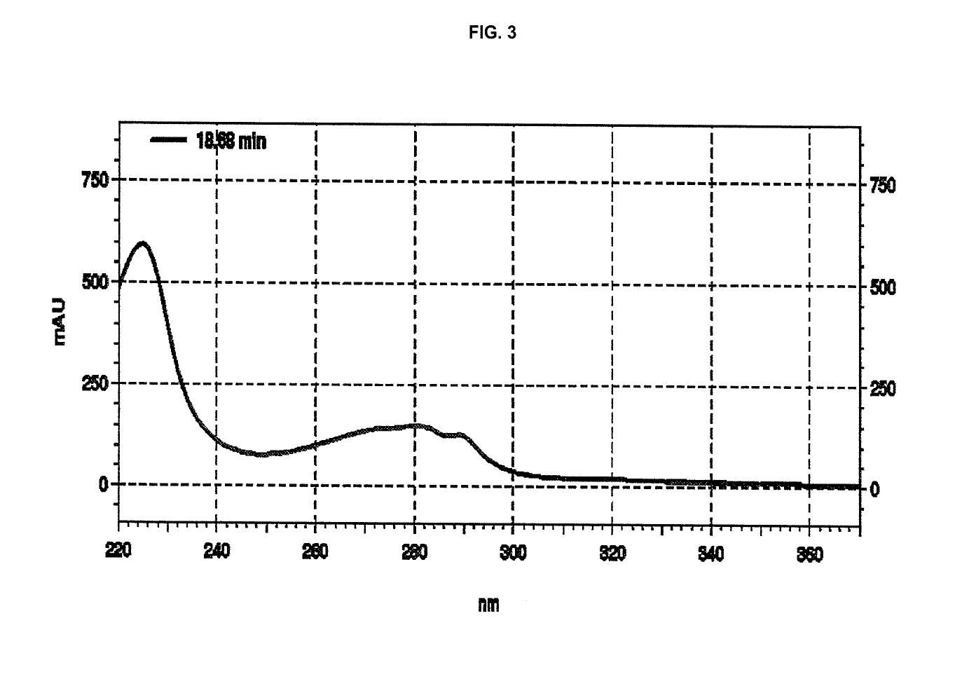

B) The U.V. spectrum of antibiotic NAI-802, performed in TFA 0.1%-acetonitrile (in ratio 50:50) with a Shimadzu Diode Array detector SPD-M10A VP (Shimadzu Corporation, Japan) during a HPLC analysis, exhibits two maxima at 225 and 280 nm. UV spectrum is reported in FIG. 3

C) ¹H-NMR and 2D experiments are recorded in the mixtures CD₃CN/D₂O (1/1) with and without the addition of 50 μL of H₂O at 25° C. on a Bruker AMX 600 or 400 spectrometers. If necessary a water suppression sequence is applied.

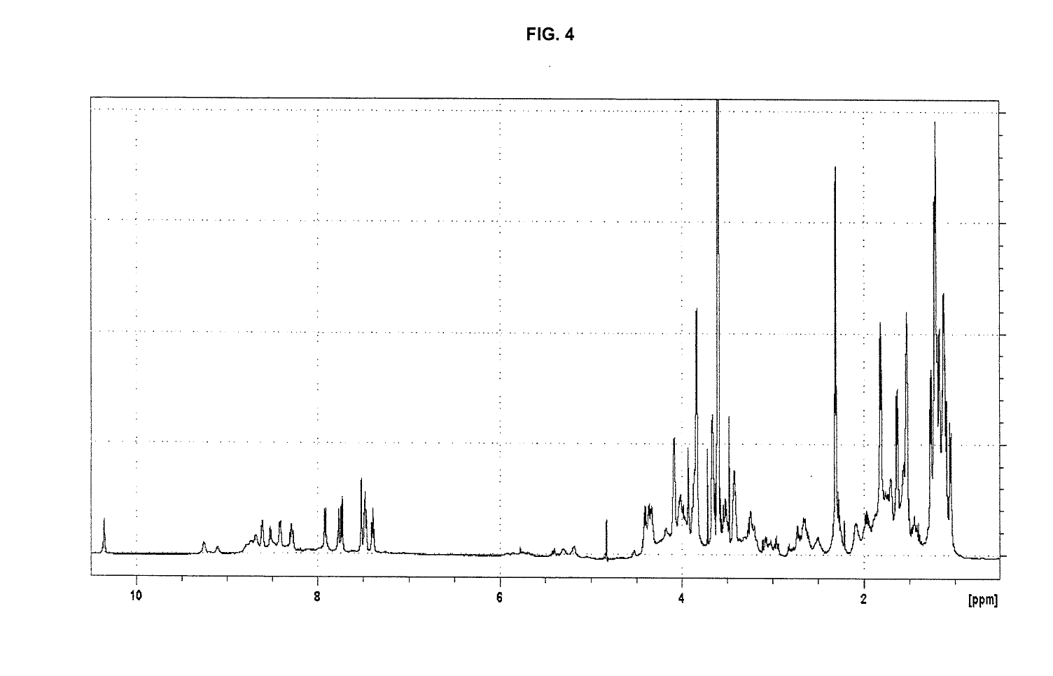

¹H NMR spectrum of antibiotic NAI-802 exhibits the following groups of signals [δ=ppm; multiplicity; (attribution)]: 1.04 d (CH₃), 1.08-1.21 overlapped (8 CH₃), 1.25 d (CH₃), 1.52-1.53 overlapped (2 CH₃), 1.63 d (CH₃), 1.7 d (CH₃), 1.81 d (CH₃), 1.44-3.63 (peptidic beta CH and CH₂), 3.91-5.29 (peptidic alpha CH and CH₂), 7.39-10.35 (aromatic CH's and peptidic NH's). The ¹H-NMR spectrum of NAI-802 is reported in FIG. 4.

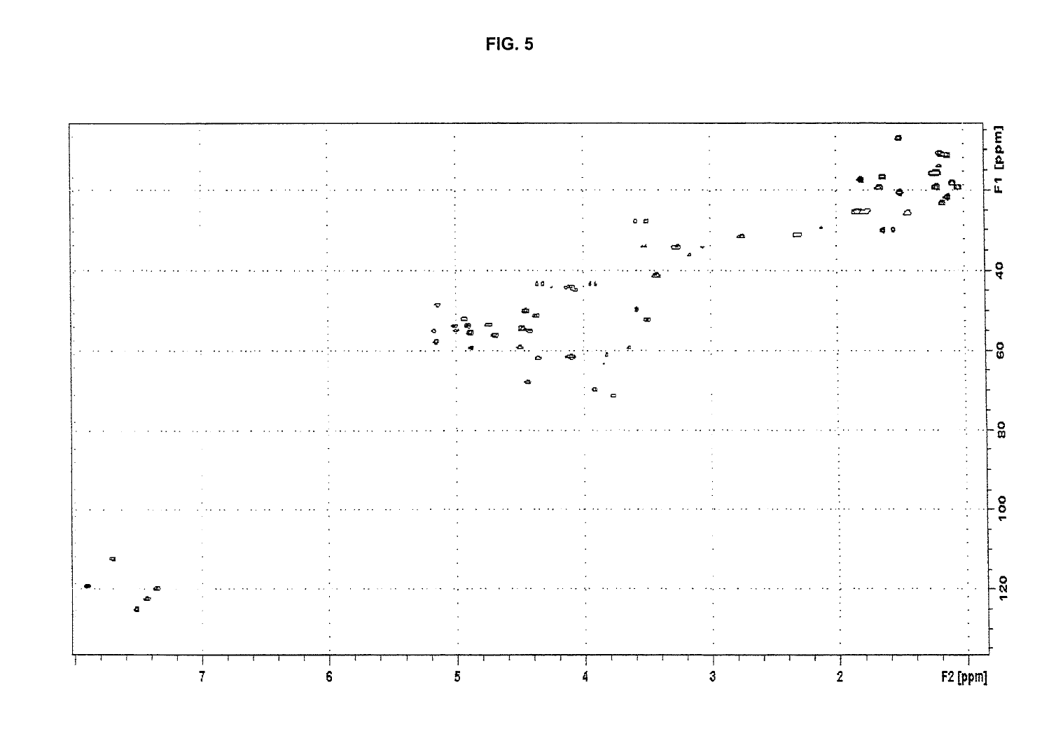

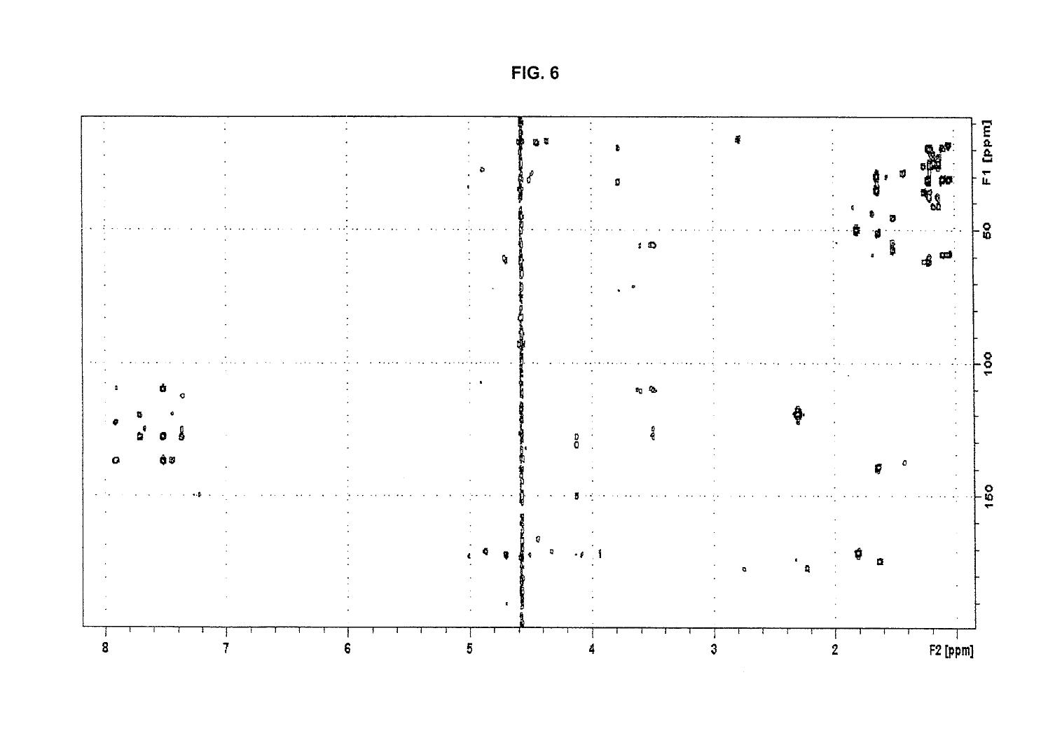

NAI-802 exhibits the following 13C groups of signals [δ=ppm; (attribution)]: 7.2-23.1 (aliphatic CH₃'s), 25-41.4 (peptidic beta CH and CH₂), 52.5 (peptidic beta CH₂), 43.6-61.7 (peptidic alpha CH and CH₂), 109.4-157 (aromatic and quaternary carbons), 171-175 (peptidic carbonyls). HSQC and HMBC spectra of NAI-802 are reported in. FIG. 5 and FIG. 6.

FIG. 5 represents the HSQC spectrum recorded in the mixture Acetonitrile-d₃:D20 at 25° C. on a Bruker AMX 600 spectrometer. Automatic peak list as obtained with Bruker software (Topspin ver. 3.0.b.8) is reported in the following table.

| δ 1H | δ 13C | |
|---|---|---|
| (ppm) | (ppm) | Intensity |
| 1.04 | 18.92 | 2.12E+07 |
| 1.09 | 17.95 | 2.26E+07 |
| 1.12 | 21.51 | 2.36E+07 |
| 1.13 | 10.99 | 1.76E+07 |
| 1.16 | 22.80 | 2.41E+07 |
| 1.18 | 10.51 | 1.76E+07 |
| 1.19 | 13.65 | 5.93E+06 |
| 1.20 | 15.36 | 2.12E+07 |
| 1.21 | 18.92 | 3.27E+07 |
| 1.25 | 15.36 | 2.37E+07 |
| 1.44 | 25.55 | 6.06E+06 |
| 1.50 | 20.38 | 2.26E+07 |
| 1.50 | 6.95 | 2.14E+07 |
| 1.55 | 29.60 | 1.14E+07 |
| 1.63 | 16.33 | 2.25E+07 |
| 1.63 | 29.76 | 2.63E+07 |
| 1.66 | 19.08 | 1.95E+07 |
| 1.75 | 25.07 | 5.73E+06 |
| 1.80 | 40.92 | 3.14E+06 |
| 1.80 | 17.14 | 2.44E+07 |
| 1.81 | 31.70 | 3.18E+06 |
| 1.84 | 25.07 | 1.12E+07 |
| 1.97 | 40.92 | 3.36E+06 |
| 1.97 | 28.95 | 3.72E+06 |
| 2.10 | 37.20 | 3.71E+06 |
| 2.12 | 29.11 | 4.26E+06 |
| 2.31 | 30.89 | 6.53E+06 |
| 2.33 | 35.58 | 3.42E+06 |
| 2.35 | 24.91 | 3.04E+06 |
| 2.55 | 24.91 | 3.14E+06 |
| 2.75 | 31.38 | 9.93E+06 |
| 2.99 | 34.42 | 3.52E+06 |
| 3.05 | 33.97 | 4.21E+06 |
| 3.16 | 35.91 | 5.25E+06 |
| 3.25 | 33.80 | 7.54E+06 |
| 3.43 | 41.08 | 1.73E+07 |
| 3.46 | 34.42 | 3.48E+06 |
| 3.49 | 27.66 | 4.41E+06 |
| 3.50 | 33.64 | 4.64E+06 |
| 3.50 | 51.92 | 7.95E+06 |
| 3.54 | 33.64 | 4.23E+06 |
| 3.58 | 49.33 | 1.03E+07 |
| 3.59 | 27.66 | 5.22E+06 |
| 3.64 | 58.90 | 5.93E+06 |
| 3.69 | 56.34 | 2.76E+06 |
| 3.82 | 60.76 | 5.25E+06 |
| 3.92 | 43.23 | 2.36E+06 |
| 4.05 | 44.64 | 5.19E+06 |
| 4.10 | 43.83 | 8.96E+06 |
| 4.10 | 61.30 | 1.76E+07 |
| 4.13 | 44.00 | 6.20E+06 |
| 4.25 | 44.16 | 4.23E+06 |
| 4.34 | 43.21 | 2.53E+06 |
| 4.35 | 61.63 | 5.93E+06 |
| 4.37 | 50.95 | 7.97E+06 |
| 4.42 | 54.83 | 7.70E+06 |
| 4.45 | 49.66 | 9.56E+06 |
| 4.47 | 54.35 | 9.58E+06 |
| 4.50 | 58.88 | 1.15E+07 |
| 4.70 | 55.97 | 1.27E+07 |
| 4.72 | 53.38 | 5.71E+06 |
| 4.75 | 53.38 | 6.16E+06 |
| 4.88 | 55.32 | 9.87E+06 |
| 4.88 | 59.04 | 1.32E+07 |
| 4.90 | 53.54 | 8.20E+06 |
| 4.93 | 51.76 | 6.84E+06 |
| 4.99 | 54.83 | 4.43E+06 |
| 5.01 | 53.54 | 8.71E+06 |
| 5.14 | 48.20 | 6.13E+06 |
| 5.16 | 57.42 | 1.26E+07 |
| 5.17 | 55.00 | 6.91E+06 |
| 7.36 | 119.54 | 8.54E+06 |
| 7.44 | 122.13 | 9.31E+06 |
| 7.52 | 124.72 | 1.70E+07 |
| 7.70 | 112.10 | 9.18E+06 |
| 7.91 | 118.89 | 9.08E+06 |

FIG. 6 represents the HMBC spectrum recorded in the mixture Acetonitrile-d₃:D20 at 25° C. on a Bruker AMX 600 spectrometer. Automatic peak list as obtained with Bruker software (Topspin ver. 3.0.b.8) is reported in the following table.

| δ 1H | δ 13C | |
|---|---|---|
| (ppm) | (ppm) | Intensity |
| 1.04 | 58.70 | 2.53E+07 |
| 1.04 | 30.69 | 3.75E+07 |
| 1.04 | 17.89 | 1.66E+07 |
| 1.09 | 58.92 | 2.27E+07 |
| 1.09 | 30.69 | 3.92E+07 |
| 1.09 | 18.97 | 1.50E+07 |
| 1.12 | 40.46 | 6.79E+06 |
| 1.12 | 23.10 | 1.11E+07 |
| 1.12 | 24.83 | 6.02E+07 |
| 1.16 | 21.36 | 8.56E+06 |
| 1.16 | 24.40 | 3.81E+07 |
| 1.17 | 40.68 | 1.03E+07 |
| 1.20 | 37.21 | 1.39E+07 |
| 1.20 | 18.97 | 1.78E+07 |
| 1.21 | 61.52 | 2.03E+07 |
| 1.21 | 30.91 | 5.15E+07 |
| 1.24 | 25.70 | 1.17E+07 |
| 1.24 | 35.47 | 2.68E+07 |
| 1.25 | 61.52 | 7.64E+06 |
| 1.42 | 28.31 | 7.95E+06 |
| 1.50 | 45.24 | 1.12E+07 |
| 1.50 | 54.79 | 5.61E+06 |
| 1.50 | 56.96 | 8.74E+06 |
| 1.56 | 29.61 | 4.24E+06 |
| 1.63 | 174.19 | 2.29E+07 |
| 1.63 | 50.88 | 2.85E+07 |
| 1.63 | 34.60 | 6.49E+07 |
| 1.64 | 29.61 | 5.88E+07 |
| 1.67 | 59.13 | 2.65E+06 |
| 1.67 | 43.50 | 4.47E+06 |
| 1.80 | 171.15 | 4.31E+07 |
| 1.80 | 49.80 | 6.11E+07 |
| 1.83 | 41.11 | 3.45E+06 |
| 1.95 | 54.14 | 2.25E+06 |
| 1.97 | 54.14 | 2.47E+06 |
| 2.23 | 176.58 | 1.31E+07 |
| 2.32 | 173.76 | 2.55E+06 |
| 2.74 | 24.83 | 2.10E+06 |
| 2.74 | 177.23 | 2.96E+06 |
| 2.78 | 15.93 | 4.68E+06 |
| 3.26 | 53.05 | 2.22E+06 |
| 3.41 | 24.83 | 2.26E+06 |
| 3.43 | 157.04 | 2.29E+06 |
| 3.43 | 25.27 | 2.13E+06 |
| 3.46 | 109.93 | 2.42E+06 |
| 3.49 | 109.72 | 6.84E+06 |
| 3.49 | 124.69 | 3.15E+06 |
| 3.49 | 127.08 | 4.38E+06 |
| 3.49 | 55.23 | 7.38E+06 |
| 3.51 | 109.06 | 3.86E+06 |
| 3.59 | 55.44 | 3.40E+06 |
| 3.59 | 110.15 | 3.95E+06 |
| 3.62 | 109.72 | 3.05E+06 |
| 3.65 | 70.64 | 2.84E+06 |
| 3.77 | 72.38 | 2.40E+06 |
| 3.77 | 18.97 | 4.49E+06 |
| 3.78 | 31.78 | 7.44E+06 |
| 3.90 | 172.45 | 2.32E+06 |
| 3.93 | 172.24 | 2.83E+06 |
| 3.93 | 170.72 | 3.11E+06 |
| 4.08 | 171.80 | 2.77E+06 |
| 4.12 | 127.52 | 3.40E+06 |
| 4.12 | 130.56 | 7.45E+06 |
| 4.12 | 149.66 | 8.22E+06 |
| 4.12 | 171.80 | 2.44E+06 |
| 4.32 | 170.94 | 3.64E+06 |
| 4.35 | 16.58 | 6.51E+06 |
| 4.36 | 169.20 | 2.10E+06 |
| 4.44 | 166.16 | 4.18E+06 |
| 4.44 | 17.02 | 9.93E+06 |
| 4.46 | 166.38 | 2.05E+06 |
| 4.47 | 28.74 | 3.20E+06 |
| 4.50 | 30.69 | 4.19E+06 |
| 4.50 | 31.13 | 4.16E+06 |
| 4.50 | 172.02 | 2.88E+06 |
| 4.69 | 18.32 | 2.11E+06 |
| 4.69 | 61.30 | 5.36E+06 |
| 4.70 | 23.96 | 2.30E+06 |
| 4.70 | 172.02 | 8.83E+06 |
| 4.87 | 170.72 | 1.05E+07 |
| 4.87 | 27.22 | 2.86E+06 |
| 4.89 | 27.22 | 3.01E+06 |
| 5.00 | 172.67 | 2.91E+06 |
| 5.16 | 173.11 | 2.04E+06 |
| 7.35 | 112.10 | 3.84E+06 |
| 7.37 | 125.35 | 3.96E+06 |
| 7.37 | 127.30 | 9.24E+06 |
| 7.44 | 118.83 | 2.96E+06 |
| 7.44 | 136.85 | 5.74E+06 |
| 7.52 | 109.50 | 3.35E+07 |
| 7.52 | 136.63 | 3.27E+07 |
| 7.52 | 127.52 | 3.34E+07 |
| 7.67 | 124.48 | 4.52E+06 |
| 7.71 | 119.27 | 8.21E+06 |
| 7.72 | 127.52 | 9.83E+06 |
| 7.91 | 109.06 | 3.00E+06 |
| 7.91 | 122.09 | 8.94E+06 |
| 7.91 | 136.42 | 1.39E+07 |

D) HPLC data: NAI-802 shows a retention time of 18.6 minutes when analysed with the HPLC method 1 as above described. NAI-802 shows a retention time of 4.1 minutes when analysed with HPLC method 2 as above described.

### Physico-Chemical Characteristics of Antibiotic Ala-NAI-802

(compound of formula (I) wherein X is Ala, Y is —S(O), Z is OH)

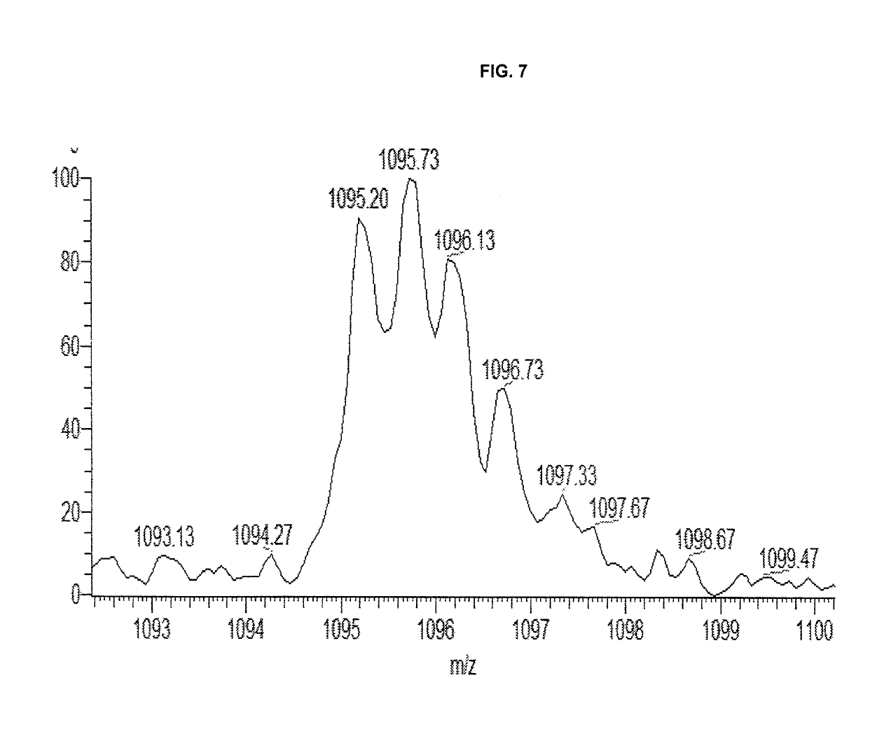

A) Mass spectrometry: in MS experiments on a Thermofinnigan LCQ deca instrument fitted with an electrospray source, using Thermofinnigan calibration mix, antibiotic Ala-NAI-802 gives a doubly protonated ion at 1095 m/z. MS/MS analysis of the double charged ion is performed with the observed main fragmentations: mono charged 745, 1445, 1856 and double charged 1086 m/z. The electrospray conditions are: Spray Voltage: 4.7 kV; Capillary temperature: 220° C.; Capillary Voltage: 3 V; Infusion mode 10 μL/min. Spectra are recorded from a 0.2 mg/mL solution in methanol/water 80/20 (v/v) with trifluoracetic acid 0.1% and are reported in FIG. 7 (full-scan low resolution spectrum).

B) The U.V. spectrum of antibiotic Ala-NAI-802, performed in TFA 0.1%-acetonitrile (in ratio 50:50) with a Shimadzu Diode Array detector SPD-M10A VP (Shimadzu Corporation, Japan) during a HPLC analysis, exhibits two maxima at 225 and 280 nm.

C) HPLC data: Ala-NAI-802 shows a relative retention time 1.025 in respect of NAI-802 when analysed with the HPLC method 2 as above described.

### Physico-Chemical Characteristics of Antibiotic Deoxy-NAI-802

(compound of formula (I) wherein X is NH₂, Y is S, Z is OH)

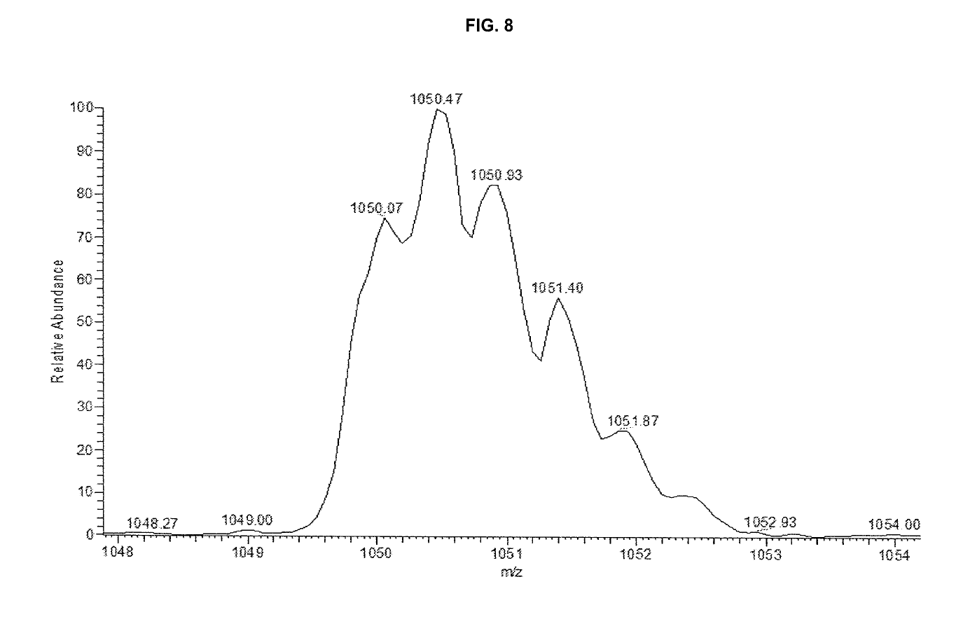

A) Mass spectrometry: in MS experiments on a Thermofinnigan LCQ deca instrument fitted with an electrospray source, using Thermofinnigan calibration mix, antibiotic Deoxy-NAI-802 gives a doubly protonated ion at 1051 m/z. The electrospray conditions are: Spray Voltage: 4.7 kV; Capillary temperature: 220° C.; Capillary Voltage: 3 V; Infusion mode 10 μL/min. Spectra are recorded from a 0.2 mg/mL solution in methanol/water 80/20 (v/v) with trifluoracetic acid 0.1% and are reported in FIG. 8 (full-scan low resolution spectrum).

B) The U.V. spectrum of antibiotic Deoxy-NAI-802, performed in TFA 0.1%-acetonitrile (in ratio 50:50) with a Shimadzu Diode Array detector SPD-M10A VP (Shimadzu Corporation, Japan) during a HPLC analysis, exhibits two maxima at 225 and 280 nm.

C) HPLC data: Deoxy-NAI-802 shows a relative retention time 1.09 in respect of NAI-802 when analysed with the HPLC method 1 as above described.

According to the preparation of NAI-802, as above described, compounds were isolated and characterized as follows.

### Determination of “Acid Resistant” Aminoacids in Antibiotic NAI-802

Acid labile amino acids are not detectable with this approach. The hydrolysate of NAI-802 was studied by HPLC-MS analysis, after suitable derivatization, in comparison with a mixture of standard amino acids similarly derivatized. Antibiotic NAI-802 was submitted to complete acidic hydrolysis (HCl 6N, 160° C., 5 minutes, microwaves). The hydrolyzed sample was treated with 4-[4-isothiocyanate-phenyl]azo-N,N-dimethyl aniline and triethylamine in water:acetonitrile 1:1. The reaction mixture was stirred 2 hours at 60° C. and extracted with petroleum ether:methylen chloride 8:2. The organic phase was evaporated to dryness, redissolved in water:acetonitrile 1:1 (1 mL) and analyzed by HPLC-MS.

The qualitative HPLC analysis was carried out on a liquid chromatography system with simultaneous DAD and MS detection. The HPLC method had the following conditions: Column: Ascentis express Supelco RP18, 2.7μ (50×4.6 mm) Column temperature: 40° C. Flow: 1 mL/min. Phase A: Trifluoroacetic acid 0.05% in water (v/v) Phase B: Trifluoroacetic acid 0.05% in acetonitrile (v/v)

Elution Program

| Time | | | | | |
|---|---|---|---|---|---|
| (min.) | 0 | 6 | 7 | 7.2 | 10 |
| % B | 5 | 95 | 100 | 5 | 5 |

MS conditions were the following: Spectrometer: Bruker Esquire3000 plus equipped with standard electrospray source: capillary temperature: 365° C.; capillary voltage: −4 kV; end plate offset: −500V; sheat gas (N₂): 50 psi.

In the LC/MS chromatograms obtained on the hydrolysate of antibiotic NAI-802, the following amino acids are identified along with other unidentified peaks: lanthionine, methyl-lanthionine, alanine, arginine, glycine, proline, tryptophan, valine, glutamic acid, leucine and isoleucine.

### Identification of N-Terminal Aminoacid in NAI-802:

1 mg of NAI-802 was dissolved in 500 μl, of Na₂CO₃ buffer (pH=8), phenylisothiocyanate (1 μL) is added and the reaction is stirred at 60° C. for 1 h. The solution was evaporated, added with trifluoroacetic acid and left at 60° C. for 1 h. HPLC-MS analysis shows that reaction is complete, with the double charged peak of m/z 1023 amu corresponding to the loss of the N-terminal Ala amino acid residue.

### EXAMPLES

### Example 1

### Fermentation Method of Aectinoplanes sp. DSM24057

Actinoplanes sp. DSM24057 is maintained on S1 plates for 2-3 weeks at 28° C. S1 is composed of (g/L): oatmeal 60, agar 18, FeSO₄×7 H₂O 0.001, MnCl₂×4 H₂O 0.001, ZnSO₄×7 H₂O 0.001 and prepared by boiling oatmeal is boiled in 1 L distilled water for 20 min, filtering it through cheesecloth, adding the remaining components adjusting volume to 1 L with distilled water and pH to 7.2 before sterilization at 121° C. for 20 min. The microbial content of one plate is scraped and inoculated into 500 mL Erlenmeyer flasks containing 100 ml of seed medium which is composed of (g/l): dextrose monohydrate 20, yeast extract 2, soybean meal 8, NaCl 1 and calcium carbonate 4. Medium is prepared in distilled water and pH adjusted to 7.3 prior to sterilization at 121° C. for 20 min. The inoculated flasks are grown at 28° C., on a rotatory shaker operating at 200 rpm. After 2-3 days, 5% of this culture is inoculated into a series of flasks containing the same medium. After 48 hours of incubation, 750 mL are transferred into 19.5 L bioreactor containing 15 L of the production medium composed of (g/L) pharmamedia 30, maize dextrin 40, yeast extract 5, glucose monohydrate 10, calcium carbonate 2, NaCl 1. The medium is prepared in deionized water and the pH adjusted to 7 before sterilization at 121° C. for 25 min, while glucose is sterilized separately and added after cooling. The fermentation is carried out at 30° C., with 400 rpm stirring and 0.4 vvm aeration. The fermenter is harvested after 90 hours of fermentation. The production of the antibiotic NAI-802 is monitored by HPLC as previously described, after extracting the whole culture broth with twice the volume of methanol and stirring for one hour.

### Example 2

### Alternative Fermentation Method of Actinoplanes sp. DSM24057

Actinoplanes sp. DSM24057 is maintained on BTT-agarplates for 2-3 weeks at 28° C. BTT-agar is composed of (g/L) glucose 10, yeast extract 1, meat extract 1, casitone 1, agar 18. Medium is prepared in distilled water and pH adjusted to 7.3 before sterilization at 121° C. for 20 min. The microbial content of one plate is scraped and inoculated into 50 mL Erlenmeyer flasks containing 15 mL of seed medium composed and prepared as described in example 1. The inoculated flasks are grown at 28° C., on a rotatory shaker operating at 200 rpm. After 3-4 days, 5% of this culture is inoculated into 500 mL Erlenmeyer flasks containing 100 mL of the same fermentation medium and grown under the same conditions. After 48 hours of incubation, 100 mL are transferred into 3 L bioreactor containing 2 L of the production medium M8 composed of (g/L): starch 20, glucose 10, yeast extract 2, casein hydrolysed 4, meat extract 4 and calcium carbonate 3. The medium is prepared in deionized water and the pH adjusted to 7.2 before sterilization at 121° C. for 25 min while glucose is sterilized separately and added after cooling. The fermentation is carried out at 30° C., with 500 rpm stirring and 0.6 vvm aeration. Sulphuric acid is added when needed to maintain pH <7.2 during the fermentation. The fermenter is harvested after 96 hours of fermentation. The production of the antibiotic NAI-802 is monitored by HPLC as described under Example 1.

### Example 3

### Recovery of Antibiotic NAI-802 (Compound of Formula (I) Wherein X is NH₂, Y is —S(O), Z is OH)

The fermentation broth described in the Example 1 is filtered with filter paper to obtain 9 L of supernatant and 3 L of concentrated mycelium. Antibiotic NAI-802 is found both in the filtrate (A) and in the mycelium (B) and these fractions were processed separately. (A) The filtered broth is concentrated to 2.5 L and then 75 ml of Diaion HP-20 polystyrenic resin were added; the mixture is stirred 5 h at room temperature and then the resin is recovered, washed with 1 L methanol: water 1:3 (v/v) and then eluted twice with 1 L methanol:water 9:1 (v/v) stirring overnight at room temperature. The pooled eluted fractions containing antibiotic NAI-802 are concentrated to small volume on a rotary evaporator and then resuspended in 10 mL water:DMF 1:1. (B) After addition of 3 L of methanol:acetic acid 9:1 (v/v), the mycelium-containing portion is stirred overnight and then filtered to obtain 3 L of cleared extract. This solution is concentrated to about 1 L solution and then stirred overnight at room temperature with 133 mL of Diaion HP-20 polystyrenic resin. The resin is then recovered, washed with 1 L methanol:water 1:3 (v/v) and then eluted twice with 1 L methanol:water 9:1 (v/v) stirring overnight at room temperature. The eluted fractions are monitored by analytical HPLC method as previously reported. The eluted fractions containing antibiotic NAI-802 are pooled, concentrated under reduced pressure and then resuspended in 10 mL water:DMF 1:1.

### Example 4

### Purification of Antibiotic NAI-802 (Compound of Formula (I) Wherein X is NH₂, Y is —S(O), Z is OH)

Crude antibiotic NAI-802 prepared as described in Example 2 under (A), is purified in two 5-mL steps by medium pressure chromatography on 86 g of reverse phase C18 RediSep Column, (Teledyne ISCO, Nebraska, USA) by using a CombiFlash Medium Pressure Chromatography System (Teledyne ISCO, Nebraska, USA) with a detection wavelength (214 nm). The resin is previously conditioned with a mixture of phase A: phase B 9:1 (v/v) and is then eluted at 60 mL/min with linear gradient from 10% to 90% of phase B in 17 min. Phase A is 50 mM ammonium formate buffer (pH 6.6) and phase B is acetonitrile. The fractions containing antibiotic NAI-802 are pooled, concentrated under vacuum and lyophilized from water, yielding 568 mg of purified antibiotic NAI-802. Crude antibiotic NAI-802, prepared as described in Example 2 under (B), is purified in two 5-mL steps as described above. The fractions containing antibiotic NAI-802 are pooled, concentrated under vacuum and lyophilized from water, yielding 907 mg of purified antibiotic NAI-802.

### Example 5

### Isolation of Deoxy-NAI-802 (Compound of Formula (I) Wherein X is NH₂, Y is S, Z is OH) from Crude NAI-802

From a sample of crude NAI-802 obtained from Example 3 (from filtrate or mycelium) deoxy-NAI-802 can be separated by HPLC as described: column: Merck Lichrospher C18 4.6 mm×100 mm; column temperature: 40° C.; flow: 1 mL/min. phase A: trifluoroacetic acid 0.1% in water (v/v) phase B: acetonitrile.

elution program

| Time (min.) | 0 | 20 | 21 | 25 | 26 | 35 |
|---|---|---|---|---|---|---|
| % B | 10 | 50 | 80 | 80 | 10 | 10 |

Under these conditions the antibiotic NAI-802 and deoxy-NIA-802 show retention times or 19.3 and 21 min respectively. A pure sample of deoxy-NAI-802 is obtained and characterized by MS analysis as previously described.

### Example 6

### Isolation of Ala-NAI-802 (Compound of Formula (I) Wherein X is Ala, Y is —S(O), Z is OH) from Crude NAI-802

From a sample of crude NAI-802 obtained from Example 3 (from filtrate or mycelium) Ala-NAI-802 can be separated by HPLC as described: column: Ascentis express Supelco RP18, 2.7μ (50×4.6 mm); column temperature: 40° C. flow: 1 mL/min. phase A: trifluoroacetic acid 0.05% in water (v/v) phase B: trifluoroacetic acid 0.05% in acetonitrile (v/v).

elution program

| Time | | | | | |
|---|---|---|---|---|---|
| (min.) | 0 | 6 | 7 | 7.2 | 10 |
| % B | 5 | 95 | 100 | 5 | 5 |

Under these analytical LC-MS conditions the antibiotic NAI-802 and ALA-NAI-802 showed retention times of 3.8 and 3.9 min respectively. A pure sample of Ala-NAI-802 is obtained and characterized by MS analysis as previously described.

### Example 7

### Preparation of Deoxy-NAI-802 (Compound of Formula (I) Wherein X is NH₂, Y is S, Z is OH)

NAI-802 (20 mg), prepared as described under Example 4, is dissolved in 10 mL buffer TRIS pH 7.8 (prepared by dissolving 2.42 g TRIS in 100 mL of water and adding 0.11 g of CaCl2. pH was brought to 7.8 by addition of 1N HCl) and is kept at 37° C. for 24 h; after that time HPLC analysis shows two major peaks with retention time of 19.3 and 21 minutes corresponding to NAI-802 and deoxy NAI-802 respectively. The observed conversion is 50%.

### Example 8

### Synthesis of NAI-802 Monoamide with Ethylendiamine (Compound of Formula (I) Wherein X is NH₂, Y is —S(O), Z is —NHCH₂CH₂NH₂) Compound 7 of Table 1

To a stirred solution of 30 mg of NAI-802, prepared as described under Example 4, in 2 ml DMF, 5.5 μL of ethylendiamine (6 eq.) and 16.5 mg of PyBOP (2 eq.) are added and the reaction is kept under stirring at room temperature for 20 minutes; after that time HPLC-MS analysis shows one major double charged peak of m/z 1080 amu corresponding to the monoamide derivative. The reaction mixture solution is then adsorbed on 4.3 g reverse-phase C18 RediSep Column, (Teledyne ISCO, Nebraska, USA) and purified by using a CombiFlash Medium Pressure Chromatography System (Teledyne ISCO, Nebraska, USA) with a detection wavelength (214 nm). The resin is previously conditioned with a mixture of phase A: phase B 9.5:0.5 (v/v) and is then eluted at 18 mL/min with linear gradient from 5% to 90% of phase B in 18 min. Phase A is TFA 0.1% and phase B is acetonitrile. The fractions containing the monoamide derivative are pooled, concentrated under vacuum and lyophilized from water, yielding 12 mg of purified NaAI-802-monoamide derivative. MS analysis shows a doubly protonated ion at m/z 1080.

### Example 9

### In Vitro Antibacterial Activity of NAI-802 and NAI-802 Monoamide with Ethylendiamine (Compound 7 of Table 1

Minimal inhibitory concentrations (MICs) for aerobic bacteria are determined by broth microdilution methodology, according to Clinical and Laboratory Standards Institute guidelines (CLSI documents M100-S16 and M27-A, NCCLS, Wayne, Pa.) using inocula of 1-5×10⁵ CFU/mL for Gram positive and negative bacteria and 1×10⁴ CFU/mL for Candida albicans.

Test results are scored after 20-24 hours of incubation at 35° C. for all tested strains, with the exception of C. albicans, which is incubated for 48 hours.

Staphylococcus aureus, enterococci, Escherichia coli and Moraxella catharralis strains are grown in Cation Adjusted Mueller Hinton (CAMHB) broth, streptococci isolates in Todd Hewitt broth and Candida albicans in RPMI-1640. All media are from Difco Laboratories, Detroit, Mich., USA. The effect of 30% bovine serum is determined under the same experimental conditions. MICs for anaerobic bacteria are determined by the broth dilution method in Brucella broth (BB) supplemented with hemin (5 μg/mL), vitamin K1 (1 μg/mL), lysed horse blood (5%) and Oxyase (1:25 v/v) (CLSI documents M11-A6, NCCLS, Wayne, Pa.).

Inocula are prepared by suspending few colonies from a 48-hours agar plate in BB to an OD₆₂₅=0.8, which is then diluted 1:10 to achieve a final suspension of about 10⁵ CFU/mL. Plates are incubated at 37° C. under anaerobic atmosphere (80% NO₂, 10% CO₂ and 10% H₂, GasPak EZ anaerobe container system, Becton Dickinson, Italy) for 48 hours.

All strains used are clinical isolates or strains from American Type Culture Collection (ATCC). The results of the tests are reported in Table II and III

Compounds NAI-802 (prepared as described under Example 4), NAI-802 monamide (prepared according to Example 8) and vancomycin (VA) are dissolved in DMSO to obtain a 10 mg/mL stock solution, and subsequently diluted in the test media to obtain working solutions. Microplates are always pre-coated with 0.02% bovine serum albumine to prevent non-specific adhesion of compounds.

TABLE II
Antimicrobial activity of antibiotic NAI-802 and NAI-802 monoamide with ethylene diamine
(Compound 7 of table 1, according to Example 8) against aerobic bacteria.

| | | Minimal Inhibitory Concentration (mg/L) | | |
|---|---|---|---|---|
| | | | NAI-802 monoamide | |
| microorganism | code | NAI-802 | with ethylendiamine | VA |
| Staphylococcus aureus Met-S ATCC6538P | 100 | 8 | 4 | 0.25 |
| +30% bovine serum | | 32 | 4 | 1 |
| Staphylococcus aureus Met-S ATCC19636 | 819 | 16 | 2 | 0.5 |
| +30% bovine serum | | 32 | 4 | 1 |
| Staphylococcus aureus Met-R | 1400 | 32 | 8 | 0.5 |
| Streptococcus pyogenes | 49 | 0.5 | ≦0.125 | 0.25 |
| Streptococcus pneumoniae | 44 | 8 | 2 | 0.50 |
| Enterococcus faecium VanS | 568 | >128 | 32 | 2 |
| +30% bovine serum | | >128 | 32 | 4 |
| Enterococcus faecium VanA | 569 | 128 | 16 | >128 |
| Enterococcus faecalis VanS | 559 | 128 | 32 | 1 |
| +30% bovine serum | | 128 | 32 | 2 |
| Enterococcus faecalis VanA | 560 | 128 | 32 | >128 |
| Moraxella catharralis | 3293 | 64 | 32 | >128 |
| Moraxella catharralis | 3294 | 64 | 32 | >128 |
| Escherichia coli | 47 | >128 | >128 | >128 |
| Candida albicans ATCC 90028 | 145 | >128 | >128 | >128 |

VA = vancomicyn, code = company's internal code for clinical isolates

TABLE III
Antimicrobial activity of antibiotic NAI-802 and NAI-802 monoamide with ethylendiamine
(Compound 7 of table 1, according to Example 8) against anaerobic bacteria

| | | Minimal Inhibitory Concentration (mg/L) | | |
|---|---|---|---|---|
| | | | NAI-802 | |
| Microorganism | Code | NAI-802 | monoamide | VA |
| Clostridium difficile | 4013 | 0.25 | ≦0.125 | 0.25 |
| Clostridium difficile | 1365 | 1 | 0.25 | 0.5 |
| Clostridium difficile | 4015 | 2 | 1 | 0.25 |
| | range | 0.25-2 | ≦0.125-1 | 0.25-0.5 |
| Clostridium butyricum | 4008 | 0.25 | 1 | 1 |
| Clostridium perfrigens | 4053 | 2 | 4 | 1 |
| Clostridium perfrigens | 3607 | 2 | 8 | 1 |
| | range | 0.25-2 | 1-8 | 1 |
| Peptstreptococcus asaccharolyticus | 521 | 1 | 0.5 | 0.5 |
| Bacteroides fragilis ATCC | 25285 | >128 | >128 | 32 |

VA = vancomicyn, code = company's internal code for clinical isolates

NAI-802 is shown herein to demonstrate antibacterial activity against Gram positive bacteria, including staphylococci and streptococci. Furthermore, it is shown herein that an ethylene diamine NAI-802 monoamide can be more antibacterial active against certain organisms than can native NAI-802.

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

The term “about” as used herein refers to a value that is +/−10% of the value to which it refers, unless otherwise defined in any particular embodiment or example. By way of a non-limiting example, the term “about 50% water” refers to an amount of water ranging from 45% to 55%.

It is to be understood that at least some of the descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present invention should not be limited to the performance of their steps in the order written, and one skilled in the art can readily appreciate that the steps may be varied and still remain within the spirit and scope of the present invention.

What is claimed is:

1. A compound of formula (I): 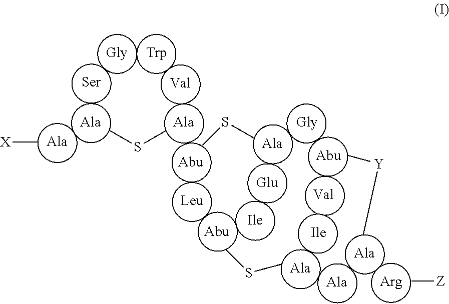
wherein X represents NH₂ or Ala; Y represents —S—, —S(O)—, —S(O)₂; Z represents OH or NR₁R₂ wherein R₁ and R₂ independently represent:
hydrogen or
an alkyl of 1 to 20 carbon atoms;
an alkenyl of 2 to 20 carbon atoms;
an alkynyl of 2 to 20 carbon atoms;
a cycloalkyl of 3 to 8 carbon atom optionally substituted by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms;
a phenyl radical optionally substituted by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms
a benzyl radical optionally substituted on the phenyl ring by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms
a naphthyl radical optionally substituted by one or two substituents selected from halo, (C1-C4)alkyl optionally substituted by 1 to 3 halogen atoms, and (C1-C4) alkoxy optionally substituted by 1 to 3 halogen atoms
a group of formula
—(CH₂)ₙOR₃
in which n represents an integer from 2 to 8 and R₃ represent
hydrogen or
(C₁-C₄) alkyl or
a cycloalkyl of 3 to 8 carbon atom optionally substituted by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms.
a phenyl radical optionally substituted by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms
a group of formula
—(CH₂)ₙNR₄R₅
in which n represents an integer from 2 to 8 and R₄ and R₅ independently represent
hydrogen or
(C₁-C₄) alkyl or
a cycloalkyl of 3 to 8 carbon atom optionally substituted by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms.
phenyl radical optionally substituted by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms
a benzyl radical optionally substituted on the phenyl ring by one or two substituents independently selected from halo, cyano, (C1-C4)alkyl optionally substituted by 1 to 3 halogen atoms, (C1-C4) alkoxy optionally substituted by 1 to 3 halogen atoms, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, (C1-C4)alkyl optionally substituted by 1 to 3 halogen atoms, and (C1-C4) alkoxy optionally substituted by 1 to 3 halogen atoms
R4 and R5 taken together represent a —(CH₂)₃, —(CH₂)₄—, —(CH₂)₂—O—(CH₂)₂, —(CH₂)₂—S—(CH₂)₂ or
R4 and R5 taken together with the adjacent nitrogen atom represent: a piperazine moiety which may be substituted in position 4 with a substituent selected from (C₁-C₄) alkyl, (C₃-C₈) cycloalkyl, pyridyl, benzyl and substituted benzyl wherein the phenyl moiety bears 1 or 2 substituents selected from chloro, bromo, nitro, (C₁-C₄) alkyl and (C₁-C₄) alkoxy.

2. A compound of formula (I) according to claim 1, wherein: Z is selected as OH.

3. A compound of formula (I) according to claim 1, wherein Z is selected as NR₁R₂, and wherein R₁ and R₂ independently represent:
an alkyl of 1 to 12 carbon atoms;
an alkenyl of 3 to 10 carbon atoms;
a cycloalkyl of 5 to 6 carbon atom optionally substituted by one or two substituents independently selected from lower alkyl of 1 to 4 carbon atoms, (C1-C4) alkoxy, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, lower alkyl of 1 to 4 carbon atoms, and (C1-C4) alkoxy.
a phenyl radical optionally substituted by one or two substituents independently selected from halo, lower alkyl of 1 to 4 carbon atoms, (C1-C4) alkoxy, phenyl, phenyl-(C1-C4)alkyl, phenoxy, phenoxy-(C1-C4)alkyl of 1 to 4 carbon atoms.
a benzyl radical optionally substituted on the phenyl ring by one or two substituents independently selected from halo, cyano, lower alkyl of 1 to 4 carbon atoms, (C1-C4) alkoxy, phenyl, phenyl-(C1-C4)alkyl, phenoxy, phenoxy-(C1-C4)alkyl of 1 to 4 carbon atoms.
a naphthyl radical optionally substituted by one or two substituents selected from halo, lower alkyl of 1 to 4 carbon atoms, and (C1-C4) alkoxy
a group of formula
—(CH₂)ₙOR₃
in which n represents an integer from 2 to 5 and R₃ represent
hydrogen or
(C₁-C₄) alkyl or
a cycloalkyl of 5 to 6 carbon atom optionally substituted by one or two substituents independently selected from halo, cyano, lower alkyl of 1 to 4 carbon atoms, (C1-C4) alkoxy, phenyl, phenyl-(C1-C4)alkyl, phenoxy, phenoxy-(C1-C4)alkyl of 1 to 4 carbon atoms.
a phenyl radical optionally substituted by one or two substituents independently selected from halo, cyano, lower alkyl of 1 to 4 carbon atoms, (C1-C4) alkoxy, phenyl, phenyl-(C1-C4)alkyl, phenoxy, phenoxy-(C1-C4)alkyl of 1 to 4 carbon atoms.
a group of formula
—(CH₂)ₙNR₄R₅
in which n represents an integer from 2 to 8 and R₄ and R₅ independently represent
hydrogen or
(C₁-C₄) alkyl or
a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-(C1-C4)alkyl, phenoxy, phenoxy-(C1-C4)alkyl wherein the phenyl and the phenyl portion of the phenyl lower-alkyl, phenoxy and phenoxy-(C1-C4)alkyl group is optionally substituted by one or two substituents selected from halo, cyano, lower alkyl of 1 to 4 carbon, and (C1-C4) alkoxy.
a phenyl radical optionally substituted by one or two substituents independently selected from halo, lower alkyl of 1 to 4 carbon atoms, (C1-C4) alkoxy, phenyl, phenyl-(C1-C4)alkyl, phenoxy, phenoxy-(C1-C4)alkyl of 1 to 4 carbon atoms.
a benzyl radical optionally substituted on the phenyl ring by one or two substituents independently selected from halo, cyano, lower alkyl of 1 to 4 carbon atoms, (C1-C4) alkoxy, phenyl, phenyl-(C1-C4)alkyl, phenoxy, phenoxy-(C1-C4)alkyl of 1 to 4 carbon atoms.
R4 and R5 taken together represent a —(CH₂)₃, —(CH₂)₄—, —(CH₂)₂—O—(CH₂)₂, —(CH₂)₂—S—(CH₂)₂ or
R4 and R5 taken together with the adjacent nitrogen atom represent: a piperazine moiety which may be substituted in position 4 with a substituent selected from (C₁-C₄) alkyl, (C₃-C₈) cycloalkyl, pyridyl, benzyl and substituted benzyl wherein the phenyl moiety bears 1 or 2 substituents selected from chloro, bromo, nitro, (C₁-C₄) alkyl and (C₁-C₄) alkoxy.

4. The compound of formula (I) according to claim 2, wherein X is NH₂, Y is —S(O) and Z is OH (NAI-802).

5. The compound of formula (I) according to claim 2, wherein X is Ala, Y is —S(O) and Z is OH (Ala-NAI-802).

6. The compound of formula (I) according to claim 2, wherein X is NH₂, Y is —S— and Z is OH (deoxy-NAI-802).

7. The compound of formula (I) according to claim 2, wherein X is Ala, Y is —S—, and Z is OH (deoxy-Ala-NAI-802).

8. The compound of formula (I) according to claim 3 wherein —NR₁R₂ has the following formula:

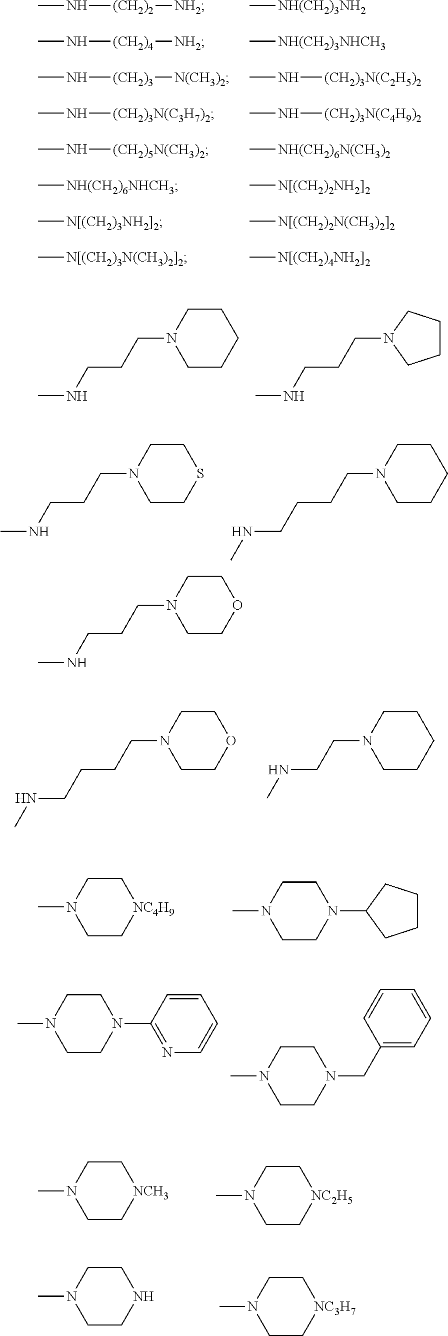

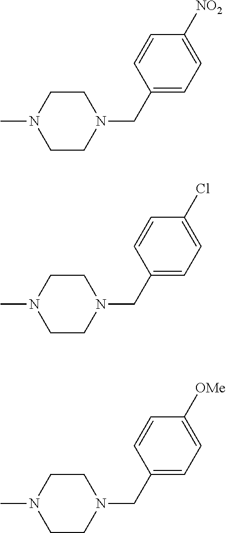 [SMILES: CN1CCN(Cc2ccc([N+](=O)[O-])cc2)CC1 ; CN1CCN(Cc2ccc(Cl)cc2)CC1 ; COc1ccc(CN2CCN(C)CC2)cc1]

9. The compound of formula (I) according to claim 3, wherein said NR₁R₂ is selected among:
TABLE 1

| | —NR₁R₂ |
|---|---|
| 1 | 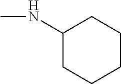 |
| 2 | 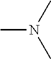 |
| 3 | 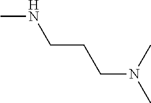 |
| 4 | 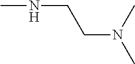 |
| 5 | 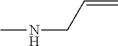 |
| 6 | 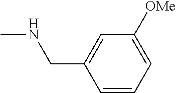 |
| 7 | 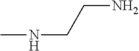 |
| 8 | 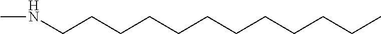 |
| 9 | 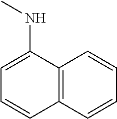 |
| 10 | 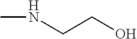 |
| 11 | 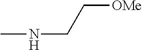 |
| 12 | 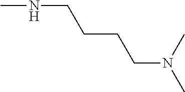 |
| 13 | 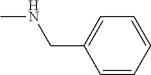 |
| 14 | 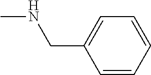 |
| 20 | 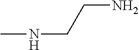 |
| 22 | 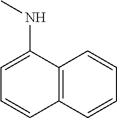 |
| 24 | 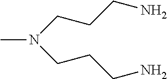 |
| 25 | 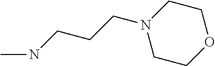 |
| 26 | 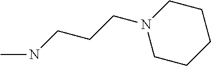 |
| 27 | 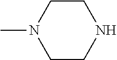 |
| 28 | 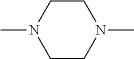 |
| 29 | 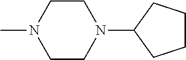 |
| 30 | 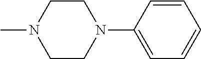 |
| 31 | 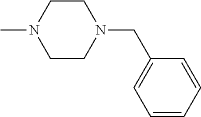 |

10. The compound of formula (I) according to claim 1 wherein X is NH₂, Y is S(O), Z is —NHCH₂CH₂NH₂.

11. A process for the preparation of a compound of formula (I) according to claim 1, comprising:
(a) cultivating Actinoplanes sp. 104802, Actinoplanes sp. 104771, or a variant or mutant thereof maintaining the ability to produce antibiotic of formula (I), under aerobic conditions, in an aqueous nutrient medium containing an assimilable source of carbon, nitrogen and inorganic salts;
(b) isolating the resulting antibiotic of formula (I) from the whole culture broth, or from the separated mycelium or from the filtered fermentation broth;
(c) purifying the isolated antibiotic of formula (I).

12. A process according to claim 11, wherein the strain Actinoplanes sp. 104802 or Actinoplanes sp. 104771 is pre-cultured.

13. The process according to claim 11, wherein the isolation of the antibiotic of formula (I) is carried out by filtering the fermentation broth and recovering the antibiotic from the filtered fermentation broth according to at least a technique selected from: extraction with a water-immiscible solvent, precipitation by adding a non-solvent or by changing the pH of the solution, absorption chromatography, partition chromatography, reverse phase partition chromatography, ion exchange chromatography, molecular exclusion chromatography, a combination of two or more of said techniques included.

14. The process according to claim 11, wherein the isolation of the antibiotic of formula (I) is carried out by separating the mycelium from the supernatant of the fermentation broth and extraction of the mycelium with a water-miscible solvent whereby, after the removal of the spent mycelium, obtaining a water-miscible solution containing the crude antibiotic, which is processed either separately or in pool with the filtered fermentation broth to recover the antibiotic NAI-802 by means of a technique selected from at least one of: extraction with a solvent, precipitation by adding a non-solvent or by changing the pH of the solution, absorption chromatography, partition chromatography, reverse phase partition chromatography, ion exchange chromatography and molecular exclusion chromatography, a combination of two or more of said techniques included.

15. The process according to claim 11, wherein X is NH₂, Y is —S(O) and Z is OH (NAI-802).

16. The process according to claim 11, comprising a condensation reaction between at least a starting compound of formula (I) wherein X is NH₂, Y is —S(O) and Z is OH, and at least a selected amine of general formula HNR₁R₂, wherein R₁ and R₂ are defined as in claim 1, in the presence of a condensing agent.

17. The process according to claim 16, wherein NR₁R₂ is selected from the group consisting of:

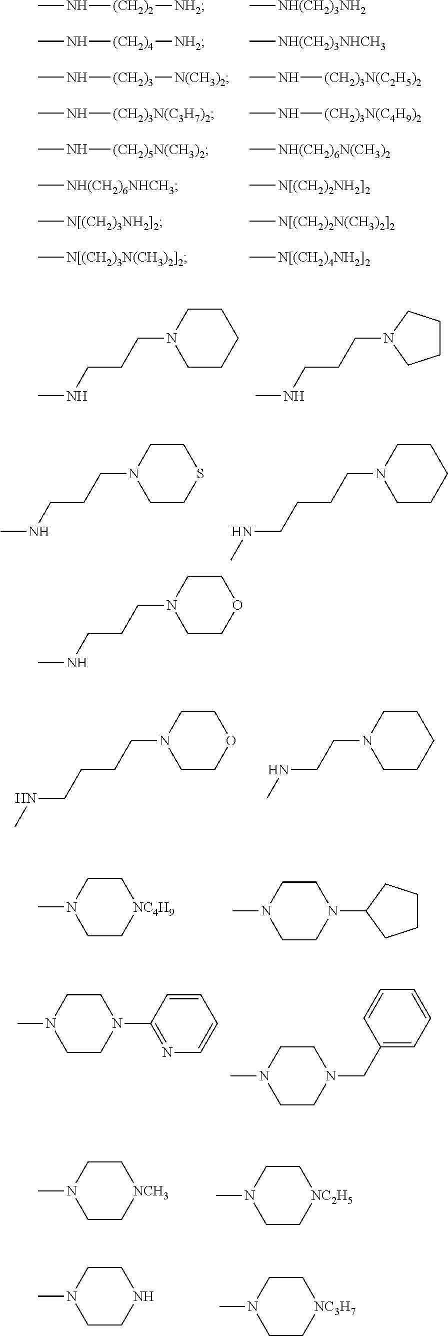

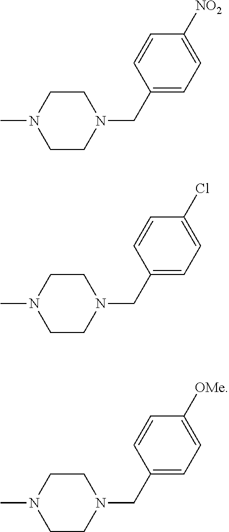 [SMILES: CN1CCN(Cc2ccc([N+](=O)[O-])cc2)CC1 ; CN1CCN(Cc2ccc(Cl)cc2)CC1 ; COc1ccc(CN2CCN(C)CC2)cc1]

18. The process of claim 16, wherein NR₁R₂ is selected from the group consisting of:
TABLE 1

| | —NR₁R₂ |
|---|---|
| 1 | 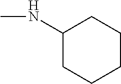 |
| 2 | 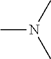 |
| 3 | 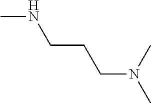 |
| 4 | 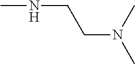 |
| 5 | 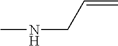 |
| 6 | 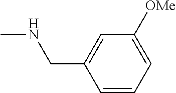 |
| 7 | 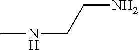 |
| 8 | 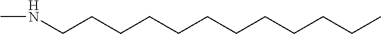 |
| 9 | 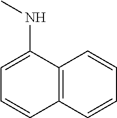 |
| 10 | 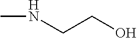 |
| 11 | 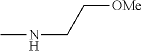 |
| 12 | 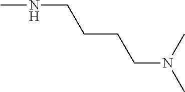 |
| 13 | 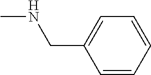 |
| 14 | 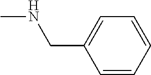 |
| 20 | 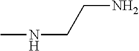 |
| 22 | 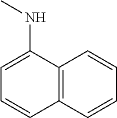 |
| 24 | 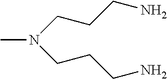 |
| 25 | 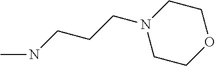 |
| 26 | 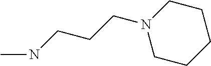 |
| 27 | 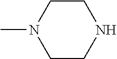 |
| 28 | 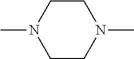 |
| 29 | 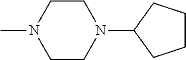 |
| 30 | 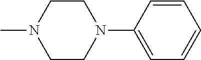 |
| 31 | 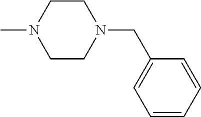 |

19. The process of claim 16, wherein said condensation reaction is carried out in the presence of at least one condensing agent and at least one solvent selected from the group consisting of organic amides, ethers of glycols, ethers of polyols, phosphoramide derivatives, sulfoxides, dimethylformamide, dimethoxyethane, hexamethyl phosphoroamide, dimethylsulphoxide, dioxane, N-methylpyrrolidone and mixtures thereof.

20. The process of claim 16, wherein said condensation reaction is carried out at a temperature ranging from 0° C. to 50° C.

21. A pharmaceutical composition comprising compound of formula (I) according to claim 1, or pharmaceutically acceptable salt thereof.

22. The pharmaceutical composition of claim 21, further comprising a pharmaceutically acceptable carrier.

23. The pharmaceutical composition of claim 21, wherein the composition is orally, topically, or parenterally administrable.

24. The pharmaceutical composition of claim 21, wherein the composition is in the forms of a capsule, a tablet, a liquid solution, a liquid suspension, an aqueous solution, an aqueous suspension, an oily solution, an oily suspension, a hydrophobic base ointment, a hydrophobic base cream, a hydrophobic base lotion, a hydrophobic base paint, a hydrophobic base powder, a hydrophilic base ointment, a hydrophilic base cream, a hydrophilic base lotion, a hydrophilic base paint, and a hydrophilic base powder.

25. A compound of formula (I) according to claim 1, or a pharmaceutically acceptable salt thereof, for use in the treatment of a bacterial infection.

26. The compound of claim 25, wherein said bacterial infection is caused by at least one of the members selected from the group consisting of enterococci, streptococci and staphylococci.

27. The compound of claim 25, wherein said bacterial infection is caused by at least one of the members selected from the group consisting of Clostridium difficile, Staphylococcus spp., Streptococcus spp, and Enterococcus spp.

28. The compound of claim 25, wherein the dosage range is comprised between 1 and 40 mg of active ingredient per Kg of body weight.

29. A biologically pure culture of the strain Actinoplanes sp. DSM104802, Actinoplanes sp. 104771, or a variant or mutant thereof maintaining the ability to produce the antibiotic of formula (I) of claim 1 when cultivated under submerged aerobic conditions in the presence of assimilable sources of carbon, nitrogen and inorganic salts.
